(19) United States
(12) Patent Application Publication
(10) Pub. No.: US 2025/0385166 A1
(43) Pub. Date: Dec. 18, 2025

(54) FRONTSIDE FEEDTHROUGH CONNECTIONS

(71) Applicant: Intel Corporation, Santa Clara, CA (US)

(72) Inventors: Saurabh Acharya, Beaverton, OR (US); Leonard Guler, Hillsboro, OR (US); Charles Henry Wallace, Portland, OR (US)

(73) Assignee: Intel Corporation, Santa Clara, CA (US)

(21) Appl. No.: 18/740,987

(22) Filed: Jun. 12, 2024

(51) Int. Cl. H01L 23/498; A61N 1/375; H01L 23/00

(52) U.S. Cl. CPC H01L 23/49811; A61N 1/3754; H01L 24/19; H01L 24/20; H01L 24/13; H01L 2224/13024; H01L 2224/19; H01L 2224/214; H01L 2224/2201; H01L 2924/13067

(57) ABSTRACT

Semiconductor devices and systems with conductive feedthroughs, and methods of forming the same, are disclosed herein. In one example, a semiconductor device includes a first interconnect, a second interconnect, and a layer between the first and second interconnects. The layer between the interconnects includes epitaxial structures and a conductive feedthrough. The conductive feedthrough extends through the layer and electrically couples the first and second interconnects, and one or more of the epitaxial structures are truncated by the conductive feedthrough.

### BACKGROUND

Backside power delivery refers to a technology for a semiconductor chip where electrical power is supplied to the active circuitry on the chip from the backside. A chip with backside power delivery technology may include feedthrough connections between the backside and frontside for power delivery to the active circuitry. In some process technologies, however, the feedthrough connections may require complex processing, may be too resistive for certain applications, and/or may limit the ability to scale the density of the activity circuitry.

### BRIEF DESCRIPTION OF THE DRAWINGS

FIG. 1 illustrates a cross-section view of a semiconductor device with a frontside feedthrough connection.

FIGS. 2A-D illustrate an example of a semiconductor device with a frontside feedthrough connection.

FIGS. 3A-D illustrate an example process flow for forming a semiconductor device with a frontside feedthrough connection.

FIG. 4 illustrates a cross-section view of an integrated circuit with frontside feedthrough connections.

FIG. 5 illustrates a flowchart for forming an integrated circuit with frontside feedthrough connections.

FIG. 6 illustrates a top view of a wafer and dies that may be included in a microelectronic assembly.

FIG. 7 illustrates a cross-sectional side view of an integrated circuit device that may be included in a microelectronic assembly.

FIGS. 8A-D illustrate perspective views of example planar, FinFET, gate-all-around, and stacked gate-all-around transistors.

FIG. 9 illustrate a cross-sectional side view of an integrated circuit device assembly that may include a microelectronic assembly.

FIG. 10 illustrates a block diagram of an example electrical device that may include a microelectronic assembly.

### DETAILED DESCRIPTION

Backside power delivery refers to a technology for semiconductor chips where electrical power is supplied to the active circuitry (e.g., transistors) on a chip from the backside rather than the frontside. A chip with backside power delivery technology typically includes backside and frontside interconnects, along with electrical feedthrough connections between the backside and frontside for power delivery and signaling to/from the active circuitry. In some process technologies, however, the feedthrough connections may be unconducive to density scaling, may require complex processing, and/or may be too resistive for certain applications.

For example, some process technologies may use deep via bars (DVB) through the semiconductor substrate to provide low-resistance paths from backside to frontside contacts, thus bypassing the active circuitry on the semiconductor substrate. However, these technologies require DVBs to be formed in areas of the semiconductor substrate with no active circuitry, which consumes valuable space that could otherwise be used for active circuitry. As a result, these technologies are not conducive to density scaling, as the ability to increase the density of active circuitry is limited by the DVBs.

As another example, some process technologies may use epitaxial feedthroughs in the active circuitry to provide high-resistance paths from backside to frontside contacts. An epitaxial feedthrough, or “epi” feedthrough, generally refers to a feedthrough connection that extends through an epitaxial structure or layer. For example, an epi feedthrough may include an epitaxial structure in the layer of active circuitry with direct backside and frontside contacts, which forms a high-resistance path between the backside and frontside contacts. In this manner, epi feedthroughs are conducive to density scaling since they can be formed directly in the active circuitry. Epi feedthroughs have a complex process flow, however, which includes both frontside and backside patterning steps with complicated etch requirements (e.g., aspect ratio, selectivity, etc.). The process flow for epi feedthroughs is also subject to various design restrictions, as the design rules require the presence of epi blocking layers or do-not-use (DNU) zones in the feedthrough regions. Further, epi feedthroughs are too resistive for some applications, such as high-current input/output (I/O) devices (e.g., analog and mixed-signal (AMS) circuits), which require low-resistance paths for current flow from backside to frontside contacts.

Thus, while DVB feedthroughs support the low-resistance paths required by high-current applications, they are not conducive to density scaling. Moreover, while epi feedthroughs are conducive to density scaling, they are too resistive for high-current applications and require very complex processing.

Accordingly, this disclosure presents embodiments of semiconductor devices and systems (e.g., integrated circuitry) with frontside feedthrough (FFT) connections that support low resistance and density scaling using a relatively simple process flow, along with methods of forming the same. For example, FFTs may be formed using a non-selective etch to form openings directly through the layer of active circuitry, which may be filled with a high-conductive, low-resistance material (e.g., metals such as tungsten (W), copper (Cu), cobalt (Co), molybdenum (Mo)). As a result, the FFTs may extend through and/or truncate certain semiconductor devices in the layer of active circuitry (e.g., portions of transistors, diodes), thus shorting those devices to provide low-resistance paths or electrical connections between backside and frontside contacts or interconnects.

Further, these feedthrough connections can be formed solely using frontside processing-no backside processing is required. Thus, while these feedthroughs provide electrical connections between the backside and frontside, they may be referred to herein as frontside feedthroughs (FFTs) since they only require frontside processing. Moreover, since the FFTs are formed directly through the active circuitry rather than through areas with no active circuitry, valuable silicon area is saved, which enables the density of active circuitry to be scaled up or increased.

The described embodiments may provide various advantages. For example, since FFTs can be formed directly through the active circuitry, they take up less space than DVBs, which enables higher density active circuitry. Further, compared to epi feedthroughs, FFTs can be formed using a more robust and simpler process while also providing better feedthrough performance (e.g., transistor performance, diode reliability). For example, FFTs can be formed using a simple process flow that uses a non-selective etch to open the feedthrough regions. As a result, the FFT process flow has less restrictive etch requirements and design rules, which eliminates the need for DNU restrictions. In particular, the FFT flow eliminates the need for epi blocking layers and DNUs in the feedthrough regions—as required by the design rules for epi feedthroughs—which reduces the overall number of DNU zones. Further, the process flow for forming FFTs only requires frontside processing-no backside processing is required. As a result, the FFT process flow enables direct frontside to backside feedthrough connections using a very simple process.

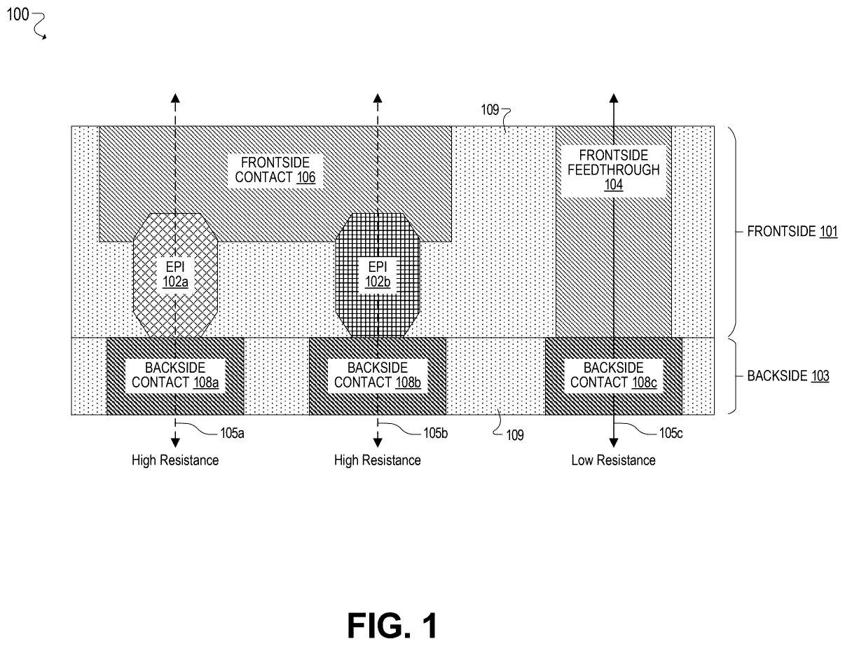

FIG. 1 illustrates a simplified cross-section view of a semiconductor device 100 with a frontside feedthrough in accordance with certain embodiments. For simplicity, the cross-section view only shows a portion of the device layer or active circuitry of device 100. In the illustrated example, device 100 includes a frontside 101 and a backside 103, along with multiple feedthrough paths or electrical connections 105a-c between the frontside 101 and backside 103. In particular, the feedthrough connections 105a-c include multiple high-resistance connections 105a-b using epitaxial (cpi) feedthroughs 102a-b, along with a low-resistance connection 105c using a frontside feedthrough 104, thus providing both high- and low-resistance paths between the frontside 101 and backside 103, as described further below.

The high-resistance feedthrough connections 105a-b include respective epitaxial (epi) feedthrough structures 102a-b electrically coupled to frontside and backside conductive contacts 106, 108a,b. In the illustrated example, the epi feedthroughs 102a-b are electrically coupled to the same frontside contact 106 and different backside contacts 108a-b. In some embodiments, the respective epi feedthroughs 102a-b may be a p-type epitaxial structure (e.g., epitaxially-grown boron-doped silicon or silicon germanium) or an n-type epitaxial structure (e.g., epitaxially-grown phosphorous-doped silicon), which may have relatively high resistance. In this manner, the high-resistance connections 105a-b have relatively high resistance since they extend through high-resistance epi feedthroughs 102a-b.

The low-resistance feedthrough connection 105c includes a frontside conductive feedthrough structure 104 electrically coupled to a backside conductive contact 108c (or alternatively, a backside metal layer or via). The frontside feedthrough structure 104 may be filled with a low-resistance conductive material, including metals such as tungsten (W), copper (Cu), cobalt (Co), and/or molybdenum (Mo). In this manner, the low-resistance connection 105c has relatively low resistance since it includes a low-resistance frontside feedthrough 104 directly coupled to a low-resistance backside contact 108c.

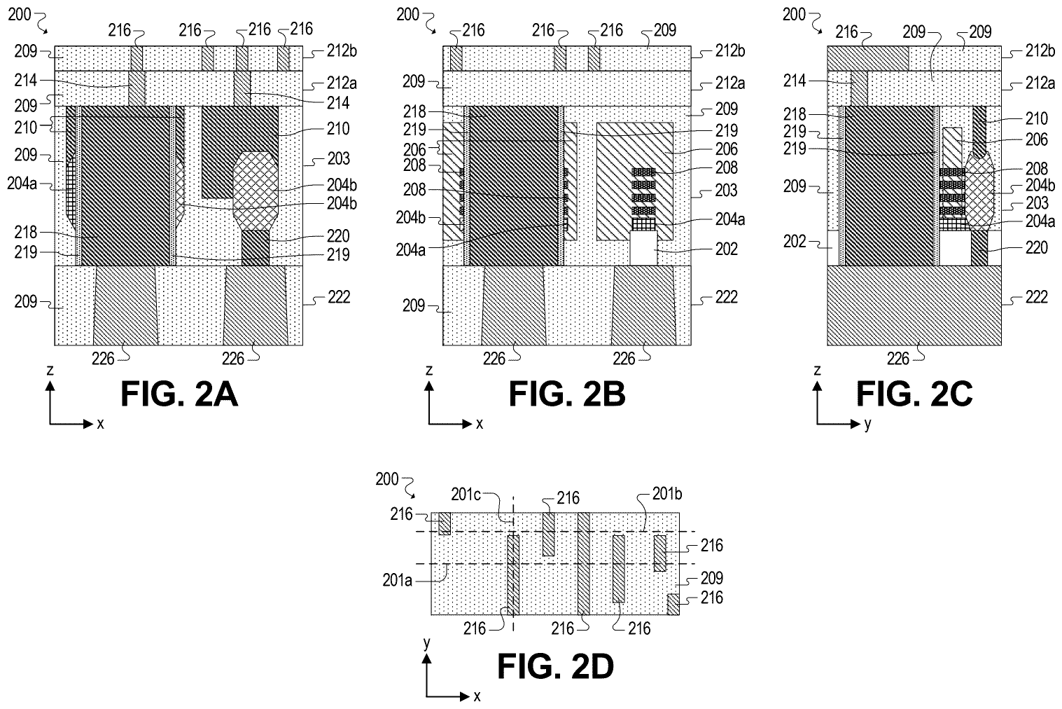

The frontside feedthrough structure 104 may be etched directly through the layer of active circuitry, thus shorting certain semiconductor devices or structures in the active circuitry (e.g., transistors, diodes) to provide a low-resistance path or electrical connection 105c between frontside 101 and backside 103 contacts or interconnects. As a result, the shorted devices may be truncated or chopped off by the frontside feedthrough 104. For simplicity, the remains of the shorted/truncated devices are not shown. However, a more detailed example of a frontside feedthrough is shown in FIGS. 2A-D, which depicts the remains of devices that are shorted/truncated by the frontside feedthrough.

The remaining areas are filled with one or more interlayer dielectrics (ILDs) 109. In actual embodiments, device 100 may include additional semiconductor devices and layers, such as transistors, diodes, metal layers with conductive traces and vias for frontside and backside interconnects, etc.

FIGS. 2A-D illustrate an example of a semiconductor device 200 with a frontside feedthrough connection 218. In particular, FIGS. 2A-C illustrate cross-section views of semiconductor device 200, and FIG. 2D illustrates a plan view of semiconductor device 200 that shows the cut lines 201a-c for the respective cross-section views of FIGS. 2A-C. In the illustrated embodiment, device 200 includes a frontside feedthrough 218 extending directly through the active circuitry 203, which is a simplified backside-to-frontside connection that can be formed using a relatively simple process flow and provides low resistance, better performance than other types of feedthroughs (e.g., epi feedthroughs), and support for density scaling, as described further below.

In the illustrated embodiment, semiconductor device 200 includes a layer of active circuitry 203, along with frontside and backside metal layers 212a-b, 222 above and below the active circuitry 203, respectively. The remaining areas of semiconductor device 200 are filled with one or more interlayer dielectrics (ILDs) 209.

The frontside and backside metal layers 212a-b, 222 include conductive traces 216, 226 and vias 214, which collectively form frontside and backside interconnects 212, 222 above and below the active circuitry 203. In various embodiments, the frontside and backside interconnects 212, 222 may be connected to the active circuitry 203 (e.g., to active devices such as transistors), may be connected to each other through the active circuitry 203 (e.g., indirectly through active devices) or through one or more feedthrough connections (e.g., frontside feedthroughs 218, epi feedthroughs, deep vias, etc.), and/or may be connected to other integrated circuit devices (not shown) (e.g., off-chip or off-package integrated circuit dies or packages).

The active circuitry 203, also referred to herein as the “device layer,” is a layer of semiconductor structures or components that collectively form one or more logic devices such as transistors. In the illustrated embodiment, the logic devices in the layer of active circuitry 203 are gate-all-around (GAA) nanoribbon transistors, where nanoribbons 208 are used as the channel of the respective transistors, and the gate 206 surrounds the nanoribbons 208 on all sides.

For example, the active circuitry 203 includes p-type and n-type epitaxial structures (cpis) 204a,b, nanoribbons 208, gates 206, frontside contacts 210, and backside contacts 220. The p-type and n-type epis 204a-b are sources and drains of the transistors, and the frontside and backside contacts 210, 220 are source/drain contacts on the frontside and backside of the source/drain epis 204a-b. The nanoribbons 208 extend between the source/drain epis 204a-b, thus coupling the source/drain epis 204a-b together and serving as the channel between them. The gates 206 are formed around the nanoribbons 208, thus surrounding them on all sides, which is a transistor design referred to as gate-all-around (GAA).

The layer of active circuitry 203 also includes the remains of a thinned semiconductor substrate 202 on which the active circuitry 203 is formed. In the illustrated embodiment, for example, the active circuitry 203 (e.g., source/drain epis 204a-b, nanoribbons 208, gates 206, frontside/backside contacts 210, 220) is formed on the frontside of a semiconductor substrate 202 (e.g., a silicon substrate), which is subsequently thinned during backside processing, such that the semiconductor device 200 only contains the remains of the thinned substrate 202.

The layer of active circuitry 203 also includes a frontside feedthrough 218, which is a low-resistance conductive (e.g., metal) feedthrough that extends directly through the active circuitry 203, thus shorting portions of the active circuitry 203, to provide a direct low-resistance connection between the frontside and backside interconnects 212, 222.

In the illustrated embodiment, for example, the frontside feedthrough 218 extends directly through some of the transistor components in the layer of active circuitry 203, such as the frontside and backside source/drain contacts 210, 220, source/drain epis 204a-b, gates 206, and nanoribbon channels 208. As a result, the frontside feedthrough 218 chops off or truncates portions of those transistor components (or replaces them altogether) and shorts the corresponding transistors. For example, if epis 204a,b are present in the feedthrough 218 region, the epis 204a,b may be chopped off or truncated by the feedthrough 218, as shown in FIG. 2A. If epis 204a,b are absent in the feedthrough 218 region, the frontside may look similar to other transistors, except the bottom of the gate 206 and the source/drain contacts 210, 220 may be shorted from the backside.

In the illustrated embodiment, the frontside feedthrough 218 also includes a dielectric liner or spacer 219 to provide isolation from the surrounding active circuitry 203 (e.g., frontside/backside contacts 210, 220, source/drain epis 204a-b, gates 206, nanoribbon channels 208).

Further, the frontside feedthrough 218 is electrically connected to both the frontside and backside interconnects 212, 222. For example, the feedthrough 218 is connected to the frontside interconnect 212 through one of the vias 214 in frontside metal layer 212a, and the feedthrough 218 is connected to the backside interconnect 222 through one of the conductive traces 226 in backside metal layer 222.

In this manner, the frontside feedthrough 218 provides a direct low-resistance connection between the frontside and backside interconnects 212, 222. In, particular, the frontside feedthrough 218 shorts the gate 206 and the source/drain contacts 210, 220 through the backside, which significantly reduces feedthrough resistance. Moreover, the resistance of the frontside feedthrough 218 can be tuned using different fill materials. For example, in some embodiments, the feedthrough 218 may be filled with a low-resistance material such as molybdenum (Mo) to lower the resistance even further.

Further, since the frontside feedthrough 218 extends directly through active circuitry 203 rather than through areas of the semiconductor substrate 202 with no active circuitry 203, valuable chip area is saved, which enables a higher density of active circuitry 203.

The respective views shown in FIGS. 2A-D will now be discussed in further detail.

FIG. 2A shows a cross-section view of semiconductor device 200 along cut line 201a (x-z plane), which is a fin cut along the frontside/backside contacts 210, 220 of the p-type and n-type source/drain epitaxial structures (epis) 204a-b. In this view, the frontside feedthrough 218 extends through one of the frontside contacts 210 and the corresponding epis 204a,b, thus chopping off or truncating portions of them, which results in partial frontside contact structures 210 and partial source/drain epitaxial structures 204a,b (e.g., epitaxial residue) on the sides of the feedthrough 218.

FIG. 2B shows a cross-section view of semiconductor device 200 along cut line 201b (x-z plane), which is another fin cut along the gates 206 (e.g., deeper along the y axis compared to FIG. 2A). In this view, the frontside feedthrough 218 extends through one of the gates 206 and the corresponding nanoribbons 208, thus chopping off or truncating portions of them, which results in partial gate structures 206 and partial nanoribbon/channel structures 208 on the sides of the feedthrough 218.

FIG. 2C shows a cross-section view of semiconductor device 200 along cut line 201c (y-z plane), which is a cut along the source/drain contacts 210, 220, epis 204a,b, nanoribbons 208, and gates 206 (e.g., perpendicular to the views of FIGS. 2A-B). In this view, the frontside feedthrough 218 extends through and replaces the source/drain contacts 210, 220, epis 204a,b, nanoribbons 208, and gate 206 that previously resided in the area occupied by the feedthrough 218.

FIG. 2D shows a top/plan view of semiconductor device 200 (x-y plane), which is overlaid with the respective cut lines 201a-c corresponding to the cross-section views of FIGS. 2A-C.

The materials used to form the respective layers and structures in device 200 may vary in different embodiments. Examples of various materials that may be used in some embodiments are provided below.

The semiconductor substrate 202 may be made of any suitable material(s), including, without limitation, silicon.

The inter-layer dielectrics (ILDs) 209 may be made of any suitable dielectric material(s), including, without limitation, silicon dioxide (SiO₂) (and/or other oxides of silicon), silicon nitride (SiN), silicon oxynitride (SiON), silicon oxycarbide (SiOC), and/or any other isolation oxides. Thus, in some embodiments, the ILDs 209 may be made of material(s) that include elements such as silicon (Si), oxygen (O), nitrogen (N), and/or carbon (C).

The gates 206 may be made of any suitable conductive or metal material(s), including, without limitation, titanium (Ti), titanium nitride (TiN), aluminum (Al), titanium aluminide (TiAl), titanium aluminum nitride (TiAlN), titanium silicon nitride (TiSiN), nickel (Ni), platinum (Pt), molybdenum (Mo), tungsten (W), cobalt (Co), and/or copper (Cu). Thus, in some embodiments, the gates 206 may be made of material(s) that include elements such as titanium (Ti), aluminum (Al), nickel (Ni), platinum (Pt), molybdenum (Mo), tungsten (W), cobalt (Co), copper (Cu), nitrogen (N), and/or silicon (Si).

The gate oxide (not shown) may be made of any suitable dielectric material(s), including, without limitation, hafnium oxide (HfO₂), silicon dioxide (SiO₂) (or other oxides of silicon), silicon oxynitride (SiON), zirconium dioxide (ZrO₂), lanthanum oxide (La₂O₃), tantalum pentoxide (Ta₂O₅), HfSiO₂, ZrSiO₂, and/or LaSiO₂. Thus, in some embodiments, the gate oxide may be made of material(s) that include elements such as hafnium (Hf), silicon (Si), oxygen (O), nitrogen (N), zirconium (Zr), lanthanum (La), and/or tantalum (Ta).

The nanoribbons 208 may be made of any suitable material(s), including, without limitation, silicon (Si), germanium (Ge), and/or silicon germanium (SiGe). Thus, in some embodiments, the nanoribbons 208 may be made of material(s) that include elements such as silicon (Si) and/or germanium (Ge).

The source/drain epitaxial structures 204a,b may be made of any suitable material(s), including, without limitation, doped silicon (Si) (e.g., phosphorous-doped Si, arsenic-doped Si, boron-doped Si) and/or doped silicon germanium (SiGe) (e.g., boron-doped SiGe with various percentages of Si and Ge). Thus, in some embodiments, the source/drain epis 204a,b may be made of material(s) that include elements such as silicon (Si), germanium (Ge), phosphorous (P), arsenic (As), and/or boron (B).

The frontside/backside source/drain contacts 210, 220 may be made of any suitable material(s), such as a contact metal, a contact trench metal, and/or a contact trench liner. For example, the source/drain contact metal may be made of any suitable conductive or metal material(s), including, without limitation, titanium (Ti), titanium nitride (TiN), aluminum (Al), titanium aluminide (TiAl), nickel (Ni), platinum (Pt), molybdenum (Mo), tungsten (W), scandium (Sc), erbium (Er), yttrium (Y), ytterbium (Yb), gadolinium (Gd), terbium (Tb), and/or dysprosium (Dy). Thus, in some embodiments, the source/drain contact metal may be made of material(s) that include elements such as titanium (Ti), aluminum (Al), nickel (Ni), platinum (Pt), molybdenum (Mo), tungsten (W), scandium (Sc), erbium (Er), yttrium (Y), ytterbium (Yb), gadolinium (Gd), terbium (Tb), dysprosium (Dy), and/or nitrogen (N). The contact trench metal may be made of any suitable conductive or metal material(s), including, without limitation, cobalt (Co), tungsten (W), molybdenum (Mo), aluminum (Al), and/or copper (Cu). The contact trench liner may be made of any suitable dielectric material(s), including, without limitation, silicon oxynitride (SiON) and/or silicon oxycarbide (SiOC). Thus, in some embodiments, the contact trench liner may be made of material(s) that include elements such as silicon (Si), oxygen (O), nitrogen (N), and/or carbon (C).

The traces 216, 226 and vias 214 may be made of any suitable conductive or metal material(s), including, without limitation, aluminum (Al), copper (Cu), cobalt (Co), molybdenum (Mo), titanium (Ti), tantalum (Ta), tungsten (W), and compounds/alloys thereof (e.g., titanium nitride (TiN)). Thus, in some embodiments, the traces 216, 226 and vias 214 may be made of material(s) that include elements such as aluminum (Al), copper (Cu), cobalt (Co), molybdenum (Mo), titanium (Ti), tantalum (Ta), tungsten (W), and/or nitrogen (N).

The frontside feedthrough 218 may be made of any suitable conductive or metal material(s), including, without limitation, tungsten (W), copper (Cu), cobalt (Co), molybdenum (Mo), and compounds/alloys thereof.

The feedthrough liner 219 may be made of any suitable dielectric material(s), including, without limitation, any of the dielectric materials referenced above with respect to ILDs 209.

It should be appreciated that the illustrated embodiment is merely shown as an example and other variations are also possible.

In various embodiments, device 200 may be implemented using other types, numbers, sizes, and/or arrangements of layers and materials than those shown and described with respect to FIGS. 2A-D. For example, device 200 may include any type and/or number of feedthroughs (e.g., frontside feedthroughs 218, epi feedthroughs, deep vias) and other semiconductor structures/components (e.g., contacts 210,220, epis 204, nanoribbons/channels 208, gates 206), any number of frontside/backside metal layers 212, 222, etc.

Moreover, certain layers or structures of device 200 may be added, replaced, omitted, and/or rearranged. For example, the substrate 202 may not be present in some embodiments, as it may be completely grinded/thinned away during fabrication. As another example, in various embodiments, the feedthrough liner 218 may or may not be included.

Further, while device 200 includes nanoribbon transistors in the illustrated embodiment, device 200 can be implemented using any type of transistors or other semiconductor devices, including nanoribbon transistors, gate-all-around (GAA) transistors, fin field-effect transistors (FinFET), planar transistors, and two-dimensional (2D) transistors (e.g., made of 2D semiconductor materials), among others.

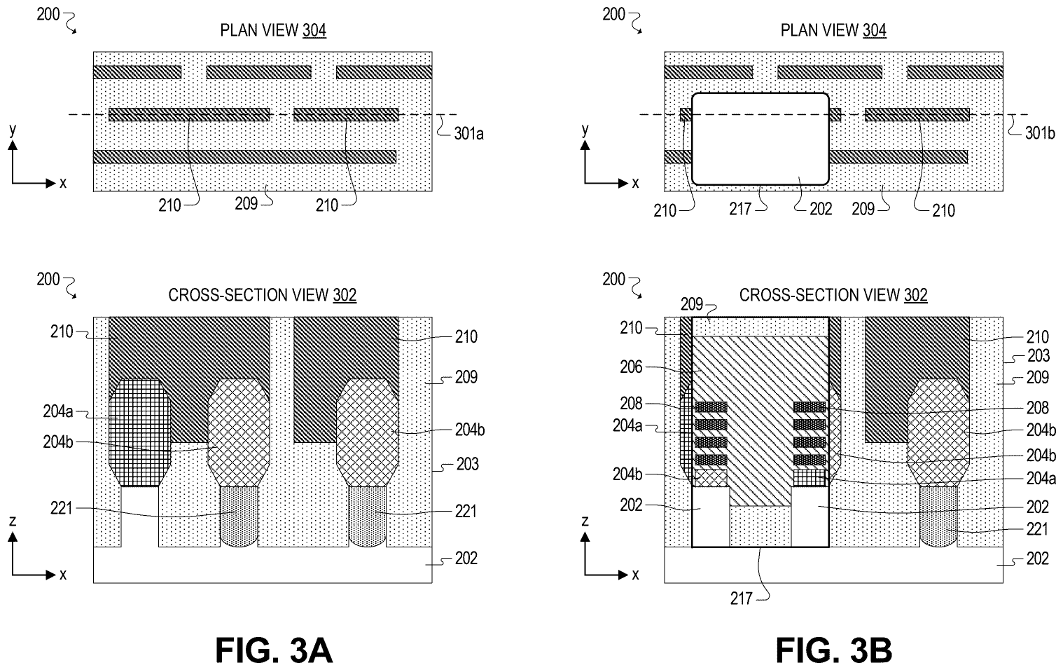

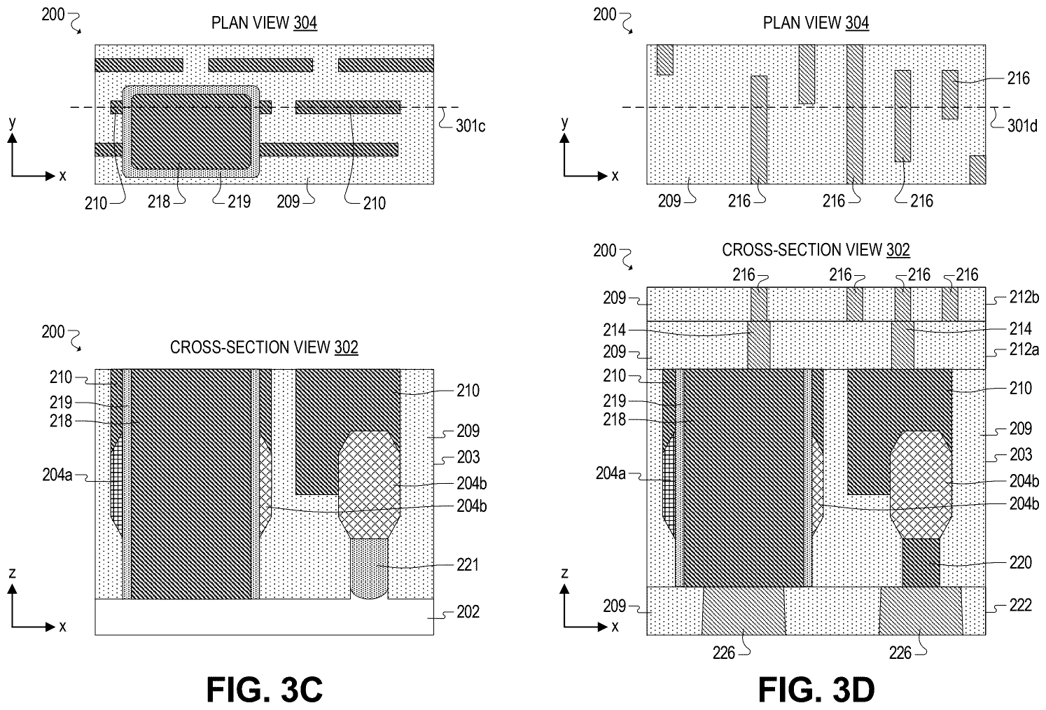

FIGS. 3A-D illustrate an example process flow for forming a semiconductor device with one or more frontside feedthrough connections. In the illustrated example, the process flow is used to form semiconductor device 200 of FIGS. 2A-D. It will be appreciated in light of the present disclosure that the illustrated process flow is only one example methodology for arriving at a semiconductor device with frontside feedthrough connections.

In the illustrated process flow, feedthroughs 218 are patterned through the active circuitry 203 on the frontside of the device 200, and frontside and backside interconnects 212, 222 are subsequently patterned above and below the feedthroughs 218, such that the respective interconnects 212, 222 are electrically coupled to each other through the frontside feedthroughs 218. The illustrated process flow uses a simple patterning loop with a non-selective directional etch to define the frontside feedthroughs 218. This process flow does not require any additional processing (e.g., lithographic patterning) for the feedthroughs 218 on the backside of the substrate/wafer 202, which simplifies the flow and may provide other benefits (e.g., increased performance/yield) since backside patterning typically has looser overlay capability than frontside patterning. This process flow also eliminates restrictions in the design rules for epi feedthroughs that require presence of epi blocking layers or do-not-use (DNU) zones in the feedthrough regions. The resulting device 200 may have various unique features, such as truncated/chopped off components in the active circuitry layer 203 (e.g., source/drain contacts 210, epis 204a-b, nanoribbons/channels 208, gate 206, etc.), as described above with respect to FIGS. 2A-D.

In the illustrated process flow, FIGS. 3A-D show cross-section (x-z plane) and plan (x-y plane) views 302, 304 of the device 200 after each step of the process flow. Further, the plan views 304 are overlaid with the cut lines 301a-d for the corresponding cross-section views 302.

In FIG. 3A, a semiconductor substrate 202 (e.g., silicon substrate) is received, and a layer 203 of active devices is formed over the substrate 202, which may also be referred to as the device layer or active circuitry. The device layer 203 includes various semiconductor structures that collectively form one or more logic devices. In the illustrated embodiment, for example, the device layer 203 includes frontside source/drain contacts 210, p-type and n-type source/drain epitaxial structures (epis) 204a,b, nanoribbons 208, and gates 206, which collectively form gate-all-around (GAA) nanoribbon transistors. The device layer 203 also includes sacrificial dummy backside contacts 221 (e.g., made of titanium nitride (TiN)) under some of the source/drain epis 204a-b, which will subsequently be replaced with the actual backside contacts 220 during backside processing (e.g., as described below with respect to FIG. 3D). The remaining areas are filled with one or more interlayer dielectrics (ILDs) 209.

In FIG. 3B, a non-selective directional etch is used to define one or more holes 217 through the device layer 203 where the frontside feedthrough(s) 218 will be formed. For simplicity, only one feedthrough hole 217 is shown in the illustrated process flow, but actual embodiments may include any number of feedthrough holes 217 (and resulting feedthroughs 218). In the illustrated example, the feedthrough holes 217 are formed by etching away regions of the device layer 203 where the frontside feedthroughs 218 will be formed. As a result, certain semiconductor structures in the feedthrough regions 217 of the device layer 203 may be partially or fully etched away (e.g., source/drain contacts 210, source/drain epis 204a,b, nanoribbons/channels 208, gates 206). For example, some semiconductor structures may be partially etched away such that only truncated portions or residue from those structures remain, while others may be completely etched away.

In FIG. 3C, a dielectric liner or spacer 219 is formed on the interior surface or wall of the respective holes 217 (e.g., before filling the holes 217 with a conductive material). The conductive frontside feedthroughs 218 are then formed by filling the holes 217 with a conductive material (e.g., metal(s) such as tungsten (W), copper (Cu), cobalt (Co), and/or molybdenum (Mo)) and polishing the surface of the conductive material.

In this manner, the frontside feedthroughs 218 extend through semiconductor structures that previously resided in the feedthrough regions of the device layer 203, thus truncating or removing those structures, and shorting the corresponding active devices (e.g., transistors) formed by those structures.

In FIG. 3D, the remaining processing is performed to complete the semiconductor device 200, such as the remaining frontside processing followed by backside processing.

For example, frontside metal layers 212a-b are formed over the device layer 203, which contain traces 216 and vias 214 that collectively form the frontside interconnect 212. For simplicity, only two frontside metal layers 212a-b are shown in the illustrated embodiment, but actual embodiments may include any number of frontside metal layers 212. In the illustrated embodiment, the frontside interconnect 212 is connected to both the frontside contacts 210 and the frontside feedthroughs 218 in the device layer 203 (e.g., through vias 214 in the first frontside metal layer 212a).

Once the frontside processing is complete, backside processing may be performed. In some embodiments, for example, a carrier substrate (not shown) is attached to the frontside (e.g., top) of the device 200, the device 200 is flipped, and the original substrate 202 is grinded/thinned to expose connections to the backside (e.g., dummy backside contacts 221, frontside feedthroughs 218, and any other connections, feedthroughs, vias, etc.). The remaining backside processing is then performed, such as forming the backside source/drain contacts 220 and the backside interconnect 222.

For example, in embodiments where dummy backside contacts 221 are formed during frontside processing, the dummy backside contacts 221 may be removed (e.g., etched away) and replaced with the actual backside contacts 220. Alternatively, if dummy backside contacts 221 are not formed during frontside processing, the backside contacts 220 may be patterned from scratch under/below the source/drain epis 204a,b during backside processing.

The backside metal layers 222 are then formed under the device layer 203, which contain traces 226 and vias (not shown) that collectively form the backside interconnect 222. For simplicity, only one backside metal layer 222 is shown in the illustrated embodiment, but actual embodiments may include any number of backside metal layers 222. In the illustrated embodiment, the backside interconnect 212 is connected to both the backside contacts 220 and the frontside feedthroughs 218 in the device layer 203 (e.g., through traces 226 in the first backside metal layer 222).

In this manner, the frontside and backside interconnects 212, 222 are electrically coupled to each other through the frontside feedthroughs 218 that extend through the device layer 203.

At this point, any remaining backside or backend processing may be performed to complete the device 200 (e.g., inter-layer dielectric (ILD) 209 filling, planarization, interconnect bump formation, etc.).

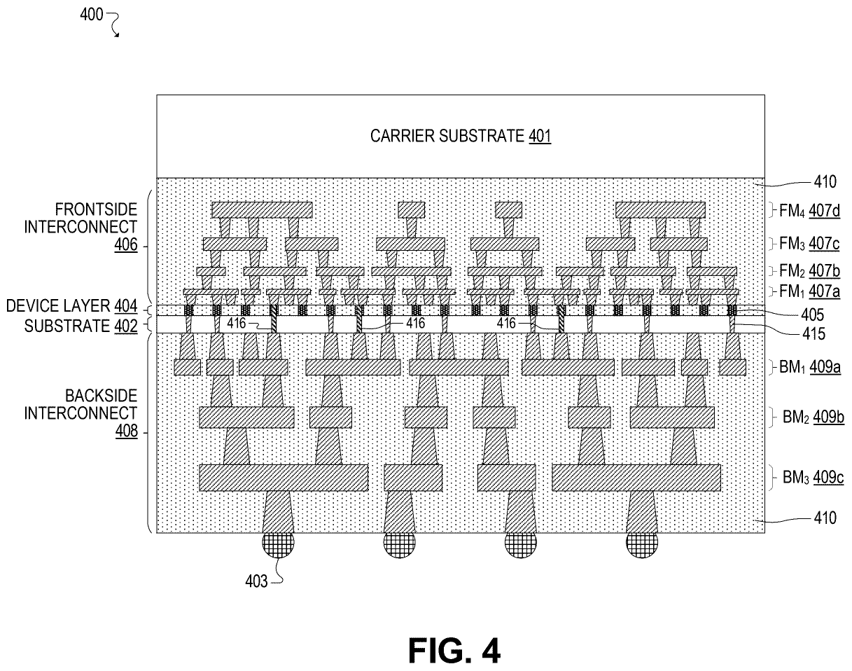

FIG. 4 illustrates a cross-section view of an integrated circuit (IC) 400 with frontside feedthrough connections in accordance with certain embodiments. In the illustrated embodiment, for example, IC 400 includes a layer 404 of logic devices 405 (e.g., transistors) with frontside feedthrough connections 416 extending through some of the logic devices 405. In various embodiments, the frontside feedthrough connections 416 may be implemented using the embodiments described throughout this disclosure.

In the illustrated embodiment, IC 400 includes a thinned silicon substrate 402, a device layer 404 over the silicon substrate 402, a frontside interconnect 406 over the device layer 404, and a backside interconnect 408 under both the device layer 404 and the silicon substrate 402. IC 400 also includes a carrier substrate 401 attached above the frontside interconnect 406 for structural support, along with conductive (e.g., metal) bumps 403 on the bottom surface to electrically couple IC 400 with another electronic device (e.g., an IC package, another IC die/chip, etc.). In some embodiments, the silicon substrate 402 may be thinned during processing to the extent that no or only limited portions of the substrate 402 remain in the completed IC 400.

The device layer 404 includes one or more logic devices 405, such as transistors and/or other semiconductor devices (e.g., nanoribbon transistors, gate-all-around (GAA) transistors, fin field-effect transistors (FinFET), planar transistors, two-dimensional (2D) transistors). Further, IC 400 includes connections 415 to the backside of certain logic devices 405, such as connections to backside source and/or drain contacts of transistors, thus providing high-resistance paths or electrical connections between the frontside and backside interconnects 406, 408.

IC 400 also includes conductive frontside feedthrough (FFT) connections 416 that extend through certain logic devices 405 in the device layer 404, thus providing low-resistance paths or electrical connections between the frontside and backside interconnects 406, 408. In some embodiments, for example, the FFT connections 416 may be implemented using the design of FFTs 104, 218 and/or the process flow of FIGS. 3A-D. For example, the FFTs 416 may be formed vertically through the device layer 404 using a highly-conductive, low-resistance material (e.g., metals such as tungsten (W), copper (Cu), cobalt (Co), molybdenum (Mo)). Further, the FFTs 416 may extend through and/or truncate certain logic devices 405 in the device layer 404, thus shorting those logic devices 405. As a result, the FFTs 416 provide low-resistance paths or electrical connections between the frontside and backside interconnects 406, 408.

In this manner, IC 400 includes both high- and low-resistance paths 415, 416 between the frontside and backside interconnects 406, 408.

The frontside interconnect 406 includes multiple frontside metal (FM) layers (FM₁₋₄) 407a-d (e.g., primarily for signaling), and the backside interconnect 408 includes multiple backside metal (BM) layers (BM₁₋₃) 409a-c (e.g., primarily for power delivery/ground connections). The remaining areas are filled with one or more inter-layer dielectrics (ILDs) 410.

In the illustrated embodiment, the device layer 404 and interconnects 406, 408 collectively implement logic circuitry with associated power (V_{DD}), ground (V_{SS}), and signal networks. In some embodiments, for example, the logic circuitry may be or may include processing circuitry, memory circuitry, storage circuitry, and/or communication circuitry.

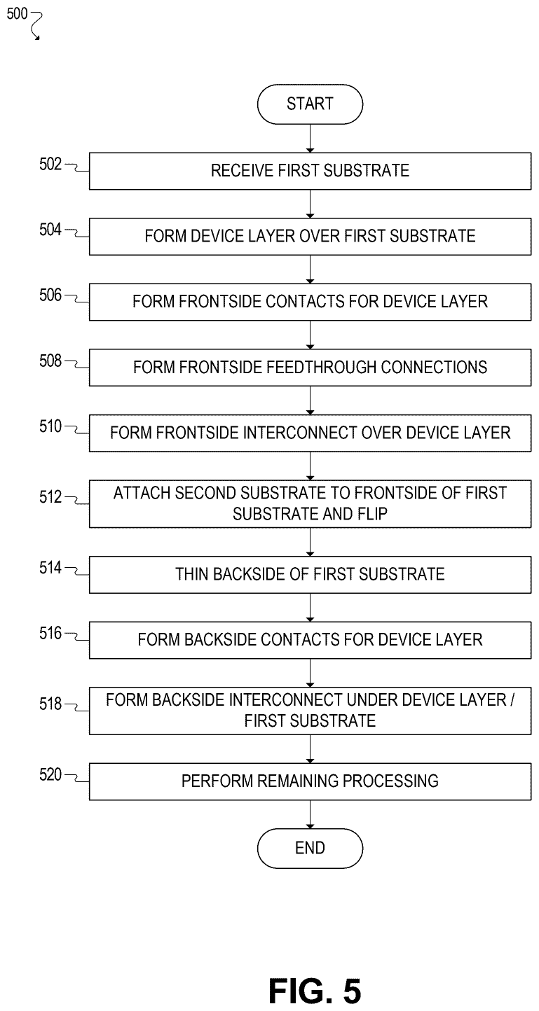

An example process flow for forming IC 400 is described below in connection with FIG. 5.

FIG. 5 illustrates a flowchart 500 for forming an integrated circuit (IC) with frontside feedthrough connections in accordance with certain embodiments. It will be appreciated in light of the present disclosure that the illustrated process flow is only one example methodology for arriving at the example IC devices shown and described throughout this disclosure (e.g., semiconductor devices 100, 200, IC 400). The steps of the illustrated process flow may be performed using any suitable semiconductor fabrication techniques. For example, film deposition—such as depositing layers, filling portions of layers (e.g., removed portions), and filling via openings—may be performed using any suitable deposition techniques, including, for example, chemical vapor deposition (CVD), metal-organic chemical vapor deposition (MOCVD), molecular beam epitaxy (MBE), atomic layer deposition (ALD), and/or physical vapor deposition (PVD). Moreover, patterning and removal—such as interconnect patterning, forming via openings, and shaping—may be performed using any suitable techniques, such as lithography-based patterning/masking and/or etching.

The illustrated process flow may be used to form one or more IC dies that respectively include a device layer, frontside and backside interconnects above and below the device layer (e.g., for signaling and power delivery), and one or more frontside feedthrough connections extending through the device layer to interconnect the frontside and backside interconnects, as described throughout this disclosure. In some embodiments, the device layer and interconnects may collectively implement logic circuitry and associated signal, power, and ground nets on the respective IC dies.

The flowchart begins at block 502 by receiving a first substrate. In some embodiments, the first substrate may be a wafer or panel and may include silicon (Si). Further, in some embodiments, the first substrate may be pre-patterned with one or more through-silicon vias (TSVs), or alternatively, the first substrate may be received without any pre-patterned TSVs and one or more TSVs may be subsequently formed in the substrate.

The flowchart then proceeds to block 504 to form a device layer over the first substrate (e.g., on the frontside of the first substrate), which may be referred to as the frontside device layer. The device layer may include one or more semiconductor devices, such as transistors (e.g., CMOS, PMOS, NMOS), to implement the logic circuitry of the respective IC dies.

In some embodiments, dummy backside contacts for the semiconductor devices may also be formed at block 504, such as dummy backside source and/or drain contacts for transistors. For example, dummy backside contacts may be formed below and/or at the bottom of the sources and/or drains of transistors to aid in forming the actual backside contacts at block 516.

The flowchart then proceeds to block 506 to form frontside conductive contacts for semiconductor devices in the device layer, such as frontside source and/or drain contacts for transistors.

The flowchart then proceeds to block 508 to form frontside feedthrough (FFT) connections in the device layer. In some embodiments, the FFTs may be formed using the process flow of FIGS. 3A-D. For example, the FFTs may be formed vertically through the device layer using a highly-conductive, low-resistance material (e.g., metals such as tungsten (W), copper (Cu), cobalt (Co), molybdenum (Mo)). Further, the FFTs may extend through and/or truncate certain semiconductor devices in the device layer, thus shorting those devices. In this manner, the FFTs will provide low-resistance paths or electrical connections between the frontside and backside interconnects formed at blocks 510 and 518, respectively.

The flowchart then proceeds to block 510 to form a first interconnect over the device layer (e.g., on the frontside of the first substrate), which may be referred to as the frontside interconnect. For example, multiple conductive (e.g., metal) layers may be formed over the device layer, along with intervening dielectric layers separating the conductive layers. The conductive layers, which may also be referred to as metal layers, may be made of one or more electrically-conductive materials that include one or more metals (e.g., any of the metals/alloys described throughout this disclosure). Further, the dielectric layers may include one or more dielectric materials (e.g., any of the dielectric materials described throughout this disclosure).

Moreover, conductive traces may be patterned (e.g., etched) in the frontside conductive layers, and vias may be formed between the conductive layers (e.g., through the intervening dielectric layers) to electrically couple traces in different conductive layers. The conductive traces and vias patterned in and between the frontside conductive layers may collectively form one or more signal, power, and/or ground nets for the logic circuitry on the respective IC dies (e.g., networks of conductive traces that provide signaling, power, and ground connections).

The signal nets (e.g., V_{IN}, V_{OUT}) may include one or more conductive traces used for signaling (e.g., electrical connections between inputs and outputs of devices/transistors in the device layer), which may also be referred to as signal traces or signal routing. Further, the signal nets in the frontside interconnect may be connected to one or more corresponding signal nets formed in the backside interconnect for off-die signal routing (e.g., as described below with respect to block 518).

The power nets (e.g., V_{DD} or V_{CC}) may include one or more conductive traces for delivering power (e.g., electrical connections between the device layer and one or more power supply terminals), which may also be referred to as power traces or power routing. Further, the power nets in the frontside interconnect may be connected to one or more corresponding power nets formed in the backside interconnect (e.g., as described below with respect to block 518).

The ground nets (e.g., V_{SS}) may include one or more conductive traces for providing ground connections (e.g., electrical connections between the device layer and one or more ground/reference terminals), which may also be referred to as ground traces or ground routing. Further, the ground nets in the frontside interconnect may be connected to one or more corresponding ground nets formed in the backside interconnect (e.g., as described below with respect to block 518).

The flowchart then proceeds to block 512 to attach or bond a second substrate to the frontside of the first substrate (e.g., over the frontside interconnect) and then flip the first substrate over. The second substrate may be referred to as a carrier substrate (e.g., a silicon carrier wafer or panel).

The flowchart then proceeds to block 514 to thin (e.g., grind) the backside of the first substrate to expose the connections (e.g., FFTs, TSVs) in the first substrate and/or any dummy backside contacts formed at block 504.

The flowchart then proceeds to block 516 to form backside conductive contacts for semiconductor devices in the device layer (e.g., on the backside of the first substrate), such as backside source and/or drain contacts for transistors. In embodiments where dummy backside contacts are formed during frontside processing (e.g., at block 504), the dummy backside contacts may be etched away and the actual backside contacts may be formed in their place.

The flowchart then proceeds to block 518 to form a second interconnect under the device layer and the first substrate (e.g., on the backside of the first substrate), which may be referred to as the backside interconnect.

For example, multiple conductive (e.g., metal) layers may be formed below the device layer and first substrate, along with intervening dielectric layers separating the conductive layers. The conductive layers, which may also be referred to as backside metal layers, may be made of one or more electrically-conductive materials that include one or more metals (e.g., any of the metals/alloys described throughout this disclosure). Further, the dielectric layers may include one or more dielectric materials (e.g., any of the dielectric materials described throughout this disclosure).

Moreover, conductive traces may be patterned (e.g., etched) in the backside conductive layers, and vias may be formed between the conductive layers (e.g., through the intervening dielectric layers) to electrically couple traces in different conductive layers. The conductive traces and vias patterned in and between the backside conductive layers may collectively form one or more backside power, ground, and/or signal nets for the logic circuitry on the respective IC dies (e.g., networks of conductive traces that provide power, ground, and/or off-chip signal routing connections). For example, the backside power and ground nets may electrically couple the corresponding frontside power and ground nets to one or more backside power supply terminals and ground terminals, respectively (e.g., through the vias in the first substrate and device layer). Moreover, the backside signal net may electrically couple the corresponding frontside signal net to one or more off-die or off-chip components (e.g., through the vias in the first substrate and device layer).

In this manner, the backside interconnect is electrically coupled to the frontside interconnect (e.g., through FFTs, vias, and/or semiconductor devices in the device layer) to provide power delivery and ground connections, along with off-die or off-chip signal routing.

The flowchart then proceeds to block 520 to perform any remaining processing, such as inter-layer dielectric (ILD) filling, planarization, interconnect bump formation, etc. In wafer or panel process flows, the completed wafer or panel may be diced to singulate the IC dies on the wafer or panel. The singulated IC dies may then be incorporated into an IC package, circuit board, electronic device, system, etc.

At this point, the flowchart may be complete. In some embodiments, however, the flowchart may restart and/or certain blocks may be repeated. For example, in some embodiments, the flowchart may restart at block 502 to continue forming one or more ICs with the same or similar design.

### Example Integrated Circuit Embodiments

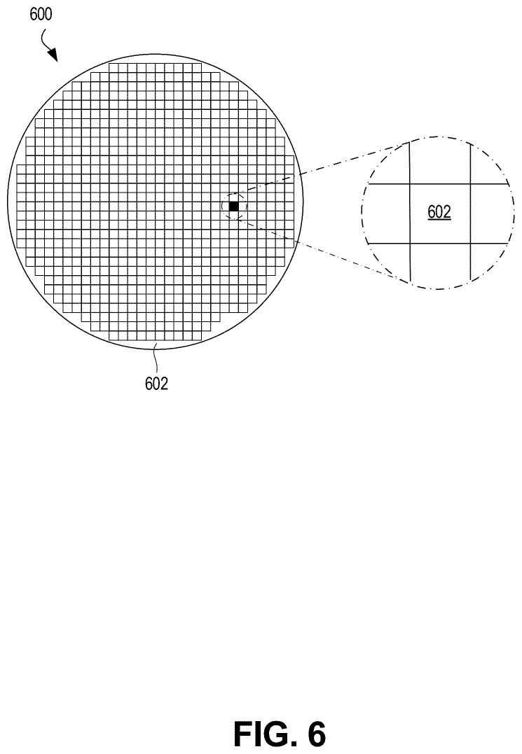

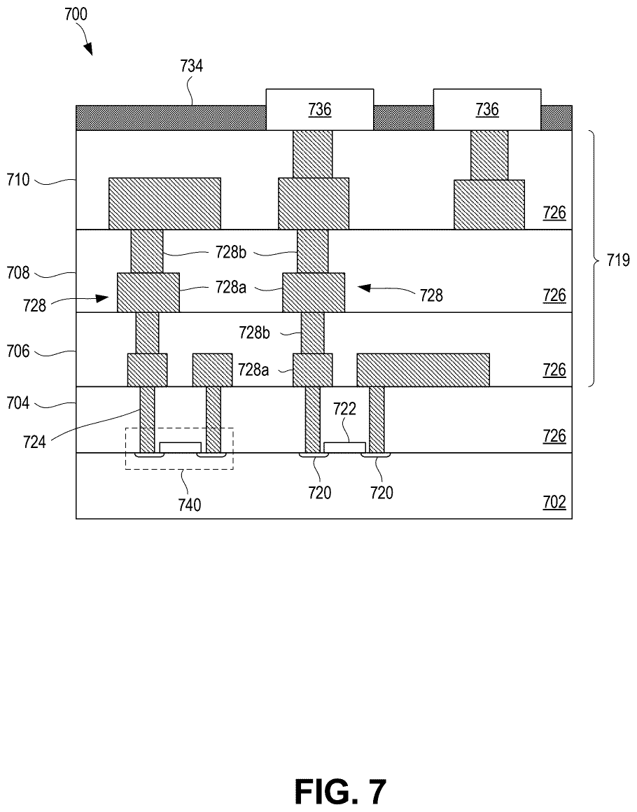

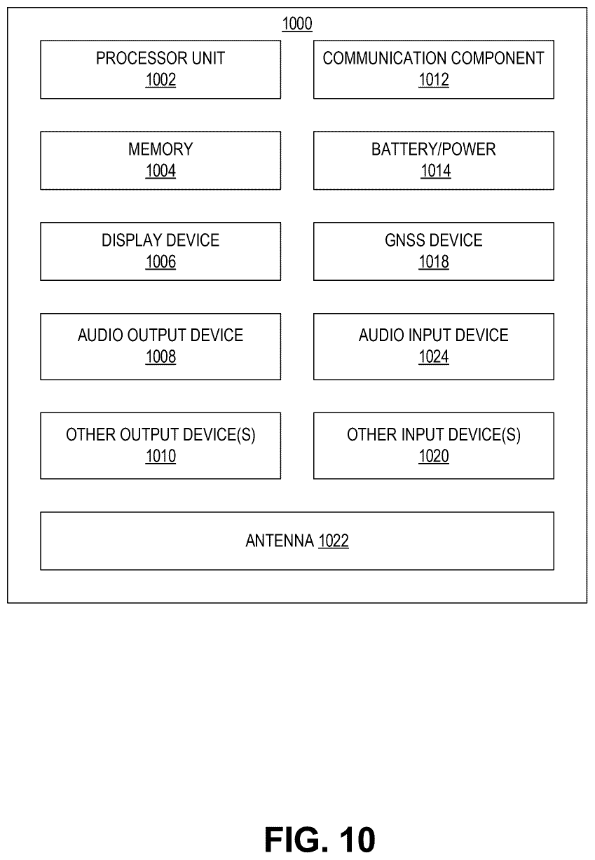

FIG. 6 is a top view of a wafer 600 and dies 602 that may be included in, or may include, any of the embodiments disclosed herein. In some embodiments, for example, the wafer 600 may include dies 602 with frontside feedthrough (FFT) connections (e.g., FFTs 104, 218, 416, semiconductor devices 100, 200, IC die 400). The wafer 600 may be composed of semiconductor material and may include one or more dies 602 having integrated circuit structures formed on a surface of the wafer 600. The individual dies 602 may be a repeating unit of an integrated circuit product that includes any suitable integrated circuit. After the fabrication of the semiconductor product is complete, the wafer 600 may undergo a singulation process in which the dies 602 are separated from one another to provide discrete “chips” of the integrated circuit product. The die 602 may be any of the dies disclosed herein. The die 602 may include one or more transistors (e.g., some of the transistors 740 of FIG. 7, discussed below), supporting circuitry to route electrical signals to the transistors, passive components (e.g., signal traces, resistors, capacitors, or inductors), and/or any other integrated circuit components. In some embodiments, the wafer 600 or the die 602 may include a memory device (e.g., a random access memory (RAM) device, such as a static RAM (SRAM) device, a magnetic RAM (MRAM) device, a resistive RAM (RRAM) device, a conductive-bridging RAM (CBRAM) device, etc.), a logic device (e.g., an AND, OR, NAND, or NOR gate), or any other suitable circuit element. Multiple ones of these devices may be combined on a single die 602. For example, a memory array formed by multiple memory devices may be formed on a same die 602 as a processor unit (e.g., the processor unit 1002 of FIG. 10) or other logic that is configured to store information in the memory devices or execute instructions stored in the memory array. Various ones of the microelectronic assemblies disclosed herein may be manufactured using a die-to-wafer assembly technique in which some dies are attached to a wafer 600 that include others of the dies, and the wafer 600 is subsequently singulated.

FIG. 7 is a cross-sectional side view of an integrated circuit device 700 that may be included in, or may include, any of the embodiments disclosed herein (e.g., semiconductor devices 100, 200, IC 400). One or more of the integrated circuit devices 700 may be included in one or more dies 602 (FIG. 6). The integrated circuit device 700 may be formed on a die substrate 702 (e.g., the wafer 600 of FIG. 6) and may be included in a die (e.g., the die 602 of FIG. 6). The die substrate 702 may be a semiconductor substrate composed of semiconductor material systems including, for example, n-type or p-type materials systems (or a combination of both). The die substrate 702 may include, for example, a crystalline substrate formed using a bulk silicon or a silicon-on-insulator (SOI) substructure. In some embodiments, the die substrate 702 may be formed using alternative materials, which may or may not be combined with silicon, that include, but are not limited to, germanium, indium antimonide, lead telluride, indium arsenide, indium phosphide, gallium arsenide, or gallium antimonide. Further materials classified as group II-VI, III-V, or IV may also be used to form the die substrate 702. Although a few examples of materials from which the die substrate 702 may be formed are described here, any material that may serve as a foundation for an integrated circuit device 700 may be used. The die substrate 702 may be part of a singulated die (e.g., the dies 602 of FIG. 6) or a wafer (e.g., the wafer 600 of FIG. 6).

The integrated circuit device 700 may include one or more device layers 704 disposed on the die substrate 702. The device layer 704 may include features of one or more transistors 740 (e.g., metal oxide semiconductor field-effect transistors (MOSFETs)) formed on the die substrate 702. The transistors 740 may include, for example, one or more source and/or drain (S/D) regions 720, a gate 722 to control current flow between the S/D regions 720, and one or more S/D contacts 724 to route electrical signals to/from the S/D regions 720. The transistors 740 may include additional features not depicted for the sake of clarity, such as device isolation regions, gate contacts, and the like. The transistors 740 are not limited to the type and configuration depicted in FIG. 7 and may include a wide variety of other types and configurations such as, for example, planar transistors, non-planar transistors, or a combination of both. Non-planar transistors may include FinFET transistors, such as double-gate transistors or tri-gate transistors, and wrap-around or all-around gate transistors, such as nanoribbon, nanosheet, or nanowire transistors.

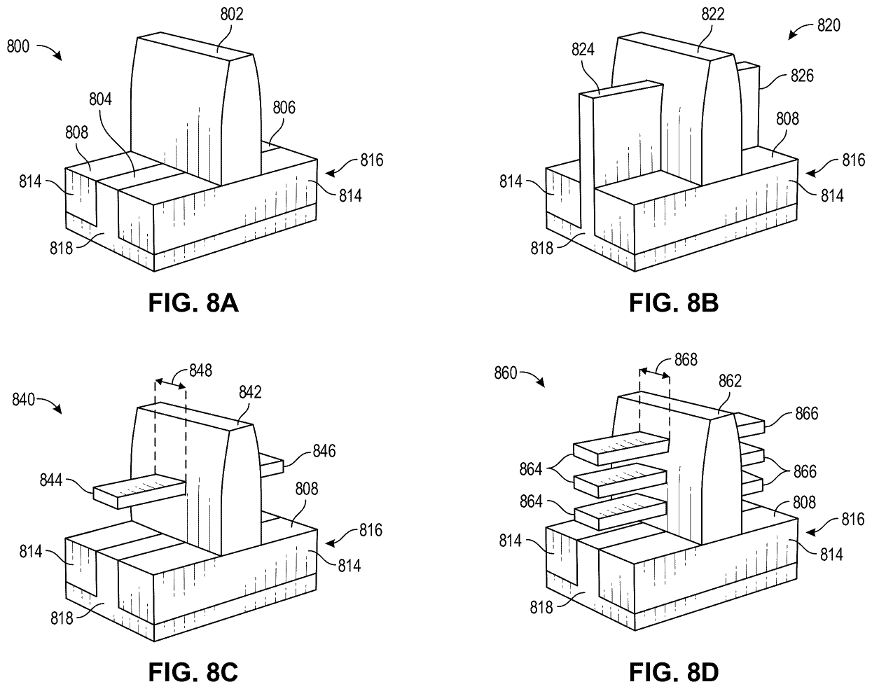

FIGS. 8A-D are simplified perspective views of example planar, FinFET, gate-all-around, and stacked gate-all-around transistors. In some embodiments, these transistors may be implemented in a device with frontside feedthrough connections (e.g., semiconductor devices 100, 200, IC die 400). The transistors illustrated in FIGS. 8A-8D are formed on a substrate 816 having a surface 808. Isolation regions 814 separate the source and drain regions of the transistors from other transistors and from a bulk region 818 of the substrate 816.

FIG. 8A is a perspective view of an example planar transistor 800 comprising a gate 802 that controls current flow between a source region 804 and a drain region 806. The transistor 800 is planar in that the source region 804 and the drain region 806 are planar with respect to the substrate surface 808.

FIG. 8B is a perspective view of an example FinFET transistor 820 comprising a gate 822 that controls current flow between a source region 824 and a drain region 826. The transistor 820 is non-planar in that the source region 824 and the drain region 826 comprise “fins” that extend upwards from the substrate surface 828. As the gate 822 encompasses three sides of the semiconductor fin that extends from the source region 824 to the drain region 826, the transistor 820 can be considered a tri-gate transistor. FIG. 8B illustrates one S/D fin extending through the gate 822, but multiple S/D fins can extend through the gate of a FinFET transistor.

FIG. 8C is a perspective view of a gate-all-around (GAA) transistor 840 comprising a gate 842 that controls current flow between a source region 844 and a drain region 846. The transistor 840 is non-planar in that the source region 844 and the drain region 846 are elevated from the substrate surface 828.

FIG. 8D is a perspective view of a GAA transistor 860 comprising a gate 862 that controls current flow between multiple elevated source regions 864 and multiple elevated drain regions 866. The transistor 860 is a stacked GAA transistor as the gate controls the flow of current between multiple elevated S/D regions stacked on top of each other. The transistors 840 and 860 are considered gate-all-around transistors as the gates encompass all sides of the semiconductor portions that extends from the source regions to the drain regions. The transistors 840 and 860 can alternatively be referred to as nanowire, nanosheet, or nanoribbon transistors depending on the width (e.g., widths 848 and 868 of transistors 840 and 860, respectively) of the semiconductor portions extending through the gate.

Returning to FIG. 7, a transistor 740 may include a gate 722 formed of at least two layers, a gate dielectric and a gate electrode. The gate dielectric may include one layer or a stack of layers. The one or more layers may include silicon oxide, silicon dioxide, silicon carbide, and/or a high-k dielectric material.

The high-k dielectric material may include elements such as hafnium, silicon, oxygen, titanium, tantalum, lanthanum, aluminum, zirconium, barium, strontium, yttrium, lead, scandium, niobium, and zinc. Examples of high-k materials that may be used in the gate dielectric include, but are not limited to, hafnium oxide, hafnium silicon oxide, lanthanum oxide, lanthanum aluminum oxide, zirconium oxide, zirconium silicon oxide, tantalum oxide, titanium oxide, barium strontium titanium oxide, barium titanium oxide, strontium titanium oxide, yttrium oxide, aluminum oxide, lead scandium tantalum oxide, and lead zinc niobate. In some embodiments, an annealing process may be carried out on the gate dielectric to improve its quality when a high-k material is used.

The gate electrode may be formed on the gate dielectric and may include at least one p-type work function metal or n-type work function metal, depending on whether the transistor 740 is to be a p-type metal oxide semiconductor (PMOS) or an n-type metal oxide semiconductor (NMOS) transistor. In some implementations, the gate electrode may consist of a stack of two or more metal layers, where one or more metal layers are work function metal layers and at least one metal layer is a fill metal layer. Further metal layers may be included for other purposes, such as a barrier layer.

For a PMOS transistor, metals that may be used for the gate electrode include, but are not limited to, ruthenium, palladium, platinum, cobalt, nickel, conductive metal oxides (e.g., ruthenium oxide), and any of the metals discussed below with reference to an NMOS transistor (e.g., for work function tuning). For an NMOS transistor, metals that may be used for the gate electrode include, but are not limited to, hafnium, zirconium, titanium, tantalum, aluminum, alloys of these metals, carbides of these metals (e.g., hafnium carbide, zirconium carbide, titanium carbide, tantalum carbide, and aluminum carbide), and any of the metals discussed above with reference to a PMOS transistor (e.g., for work function tuning).

In some embodiments, when viewed as a cross-section of the transistor 740 along the source-channel-drain direction, the gate electrode may consist of a U-shaped structure that includes a bottom portion substantially parallel to the surface of the die substrate 702 and two sidewall portions that are substantially perpendicular to the top surface of the die substrate 702. In other embodiments, at least one of the metal layers that form the gate electrode may simply be a planar layer that is substantially parallel to the top surface of the die substrate 702 and does not include sidewall portions substantially perpendicular to the top surface of the die substrate 702. In other embodiments, the gate electrode may consist of a combination of U-shaped structures and planar, non-U-shaped structures. For example, the gate electrode may consist of one or more U-shaped metal layers formed atop one or more planar, non-U-shaped layers.

In some embodiments, a pair of sidewall spacers may be formed on opposing sides of the gate stack to bracket the gate stack. The sidewall spacers may be formed from materials such as silicon nitride, silicon oxide, silicon carbide, silicon nitride doped with carbon, and silicon oxynitride. Processes for forming sidewall spacers are well known in the art and generally include deposition and etching process steps. In some embodiments, a plurality of spacer pairs may be used; for instance, two pairs, three pairs, or four pairs of sidewall spacers may be formed on opposing sides of the gate stack.

The S/D regions 720 may be formed within the die substrate 702 adjacent to the gate 722 of individual transistors 740. The S/D regions 720 may be formed using an implantation/diffusion process or an etching/deposition process, for example. In the former process, dopants such as boron, aluminum, antimony, phosphorous, or arsenic may be ion-implanted into the die substrate 702 to form the S/D regions 720. An annealing process that activates the dopants and causes them to diffuse farther into the die substrate 702 may follow the ion-implantation process. In the latter process, the die substrate 702 may first be etched to form recesses at the locations of the S/D regions 720. An epitaxial deposition process may then be carried out to fill the recesses with material that is used to fabricate the S/D regions 720. In some implementations, the S/D regions 720 may be fabricated using a silicon alloy such as silicon germanium or silicon carbide. In some embodiments, the epitaxially deposited silicon alloy may be doped in situ with dopants such as boron, arsenic, or phosphorous. In some embodiments, the S/D regions 720 may be formed using one or more alternate semiconductor materials such as germanium or a group III-V material or alloy. In further embodiments, one or more layers of metal and/or metal alloys may be used to form the S/D regions 720.

Electrical signals, such as power and/or input/output (I/O) signals, may be routed to and/or from the devices (e.g., transistors 740) of the device layer 704 through one or more interconnect layers disposed on the device layer 704 (illustrated in FIG. 7 as interconnect layers 706-710). For example, electrically conductive features of the device layer 704 (e.g., the gate 722 and the S/D contacts 724) may be electrically coupled with the interconnect structures 728 of the interconnect layers 706-710. The one or more interconnect layers 706-710 may form a metallization stack (also referred to as an “ILD stack”) 719 of the integrated circuit device 700.

The interconnect structures 728 may be arranged within the interconnect layers 706-710 to route electrical signals according to a wide variety of designs; in particular, the arrangement is not limited to the particular configuration of interconnect structures 728 depicted in FIG. 7. Although a particular number of interconnect layers 706-710 is depicted in FIG. 7, embodiments of the present disclosure include integrated circuit devices having more or fewer interconnect layers than depicted.

In some embodiments, the interconnect structures 728 may include lines 728a and/or vias 728b filled with an electrically conductive material such as a metal. The lines 728a may be arranged to route electrical signals in a direction of a plane that is substantially parallel with a surface of the die substrate 702 upon which the device layer 704 is formed. For example, the lines 728a may route electrical signals in a direction in and out of the page and/or in a direction across the page from the perspective of FIG. 7. The vias 728b may be arranged to route electrical signals in a direction of a plane that is substantially perpendicular to the surface of the die substrate 702 upon which the device layer 704 is formed. In some embodiments, the vias 728b may electrically couple lines 728a of different interconnect layers 706-710 together.

The interconnect layers 706-710 may include a dielectric material 726 disposed between the interconnect structures 728, as shown in FIG. 7. In some embodiments, dielectric material 726 disposed between the interconnect structures 728 in different ones of the interconnect layers 706-710 may have different compositions; in other embodiments, the composition of the dielectric material 726 between different interconnect layers 706-710 may be the same. The device layer 704 may include a dielectric material 726 disposed between the transistors 740 and a bottom layer of the metallization stack as well. The dielectric material 726 included in the device layer 704 may have a different composition than the dielectric material 726 included in the interconnect layers 706-710; in other embodiments, the composition of the dielectric material 726 in the device layer 704 may be the same as a dielectric material 726 included in any one of the interconnect layers 706-710.

A first interconnect layer 706 (referred to as Metal 1 or “M1”) may be formed directly on the device layer 704. In some embodiments, the first interconnect layer 706 may include lines 728a and/or vias 728b, as shown. The lines 728a of the first interconnect layer 706 may be coupled with contacts (e.g., the S/D contacts 724) of the device layer 704. The vias 728b of the first interconnect layer 706 may be coupled with the lines 728a of a second interconnect layer 708.

The second interconnect layer 708 (referred to as Metal 2 or “M2”) may be formed directly on the first interconnect layer 706. In some embodiments, the second interconnect layer 708 may include via 728b to couple the lines 728 of the second interconnect layer 708 with the lines 728a of a third interconnect layer 710. Although the lines 728a and the vias 728b are structurally delineated with a line within individual interconnect layers for the sake of clarity, the lines 728a and the vias 728b may be structurally and/or materially contiguous (e.g., simultaneously filled during a dual-damascene process) in some embodiments.

The third interconnect layer 710 (referred to as Metal 3 or “M3”) (and additional interconnect layers, as desired) may be formed in succession on the second interconnect layer 708 according to similar techniques and configurations described in connection with the second interconnect layer 708 or the first interconnect layer 706. In some embodiments, the interconnect layers that are “higher up” in the metallization stack 719 in the integrated circuit device 700 (i.e., farther away from the device layer 704) may be thicker that the interconnect layers that are lower in the metallization stack 719, with lines 728a and vias 728b in the higher interconnect layers being thicker than those in the lower interconnect layers.

The integrated circuit device 700 may include a solder resist material 734 (e.g., polyimide or similar material) and one or more conductive contacts 736 formed on the interconnect layers 706-710. In FIG. 7, the conductive contacts 736 are illustrated as taking the form of bond pads. The conductive contacts 736 may be electrically coupled with the interconnect structures 728 and configured to route the electrical signals of the transistor(s) 740 to external devices. For example, solder bonds may be formed on the one or more conductive contacts 736 to mechanically and/or electrically couple an integrated circuit die including the integrated circuit device 700 with another component (e.g., a printed circuit board). The integrated circuit device 700 may include additional or alternate structures to route the electrical signals from the interconnect layers 706-710; for example, the conductive contacts 736 may include other analogous features (e.g., posts) that route the electrical signals to external components. The conductive contacts 736 may serve as any of the conductive contacts described throughout this disclosure.

In some embodiments in which the integrated circuit device 700 is a double-sided die, the integrated circuit device 700 may include another metallization stack (not shown) on the opposite side of the device layer(s) 704. This metallization stack may include multiple interconnect layers as discussed above with reference to the interconnect layers 706-710, to provide conductive pathways (e.g., including conductive lines and vias) between the device layer(s) 704 and additional conductive contacts (not shown) on the opposite side of the integrated circuit device 700 from the conductive contacts 736. These additional conductive contacts may serve as any of the conductive contacts described throughout this disclosure.

In other embodiments in which the integrated circuit device 700 is a double-sided die, the integrated circuit device 700 may include one or more through silicon vias (TSVs) through the die substrate 702; these TSVs may make contact with the device layer(s) 704, and may provide conductive pathways between the device layer(s) 704 and additional conductive contacts (not shown) on the opposite side of the integrated circuit device 700 from the conductive contacts 736. These additional conductive contacts may serve as any of the conductive contacts described throughout this disclosure. In some embodiments, TSVs extending through the substrate can be used for routing power and ground signals from conductive contacts on the opposite side of the integrated circuit device 700 from the conductive contacts 736 to the transistors 740 and any other components integrated into the die 700, and the metallization stack 719 can be used to route I/O signals from the conductive contacts 736 to transistors 740 and any other components integrated into the die 700.

Multiple integrated circuit devices 700 may be stacked with one or more TSVs in the individual stacked devices providing connection between one of the devices to any of the other devices in the stack. For example, one or more high-bandwidth memory (HBM) integrated circuit dies can be stacked on top of a base integrated circuit die and TSVs in the HBM dies can provide connection between the individual HBM and the base integrated circuit die. Conductive contacts can provide additional connections between adjacent integrated circuit dies in the stack. In some embodiments, the conductive contacts can be fine-pitch solder bumps (microbumps).

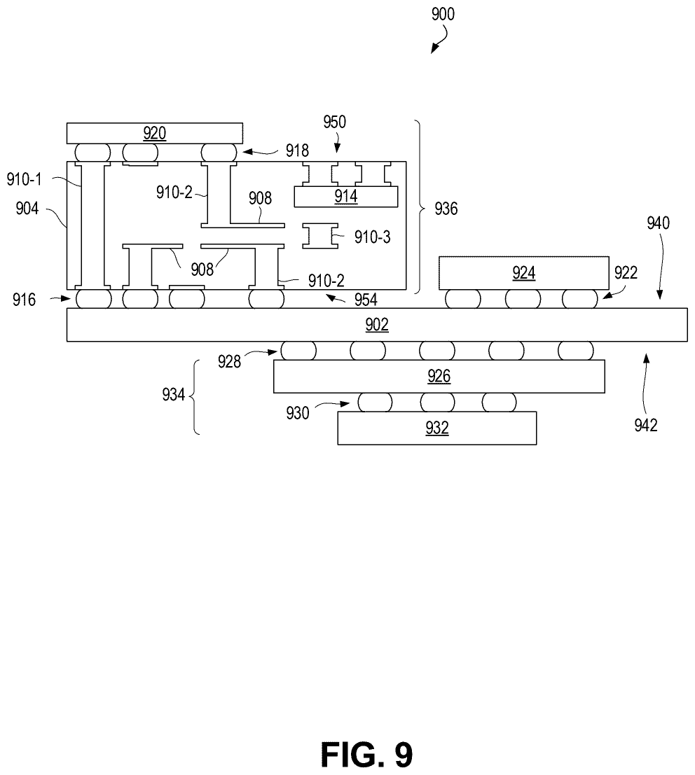

FIG. 9 is a cross-sectional side view of an integrated circuit device assembly 900 that may include any of the embodiments disclosed herein (e.g., one or more integrated circuits with frontside feedthrough connections, such as semiconductor devices 100, 200 and IC 400). In some embodiments, the integrated circuit device assembly 900 may be a microelectronic assembly. The integrated circuit device assembly 900 includes a number of components disposed on a circuit board 902 (which may be a motherboard, system board, mainboard, etc.). The integrated circuit device assembly 900 includes components disposed on a first face 940 of the circuit board 902 and an opposing second face 942 of the circuit board 902; generally, components may be disposed on one or both faces 940 and 942. Any of the integrated circuit components discussed below with reference to the integrated circuit device assembly 900 may take the form of any suitable ones of the embodiments of the microelectronic assemblies disclosed herein.

In some embodiments, the circuit board 902 may be a printed circuit board (PCB) including multiple metal (or interconnect) layers separated from one another by layers of dielectric material and interconnected by electrically conductive vias. The individual metal layers comprise conductive traces. Any one or more of the metal layers may be formed in a desired circuit pattern to route electrical signals (optionally in conjunction with other metal layers) between the components coupled to the circuit board 902. In other embodiments, the circuit board 902 may be a non-PCB substrate. The integrated circuit device assembly 900 illustrated in FIG. 9 includes a package-on-interposer structure 936 coupled to the first face 940 of the circuit board 902 by coupling components 916. The coupling components 916 may electrically and mechanically couple the package-on-interposer structure 936 to the circuit board 902, and may include solder balls (as shown in FIG. 9), pins (e.g., as part of a pin grid array (PGA), contacts (e.g., as part of a land grid array (LGA)), male and female portions of a socket, an adhesive, an underfill material, and/or any other suitable electrical and/or mechanical coupling structure. The coupling components 916 may serve as the coupling components illustrated or described for any of the substrate assembly or substrate assembly components described herein, as appropriate.

The package-on-interposer structure 936 may include an integrated circuit component 920 coupled to an interposer 904 by coupling components 918. The coupling components 918 may take any suitable form for the application, such as the forms discussed above with reference to the coupling components 916. Although a single integrated circuit component 920 is shown in FIG. 9, multiple integrated circuit components may be coupled to the interposer 904; indeed, additional interposers may be coupled to the interposer 904. The interposer 904 may provide an intervening substrate used to bridge the circuit board 902 and the integrated circuit component 920.

The integrated circuit component 920 may be a packaged or unpackaged integrated circuit product that includes one or more integrated circuit dies (e.g., the die 602 of FIG. 6, the integrated circuit device 700 of FIG. 7) and/or one or more other suitable components. A packaged integrated circuit component comprises one or more integrated circuit dies mounted on a package substrate with the integrated circuit dies and package substrate encapsulated in a casing material, such as a metal, plastic, glass, or ceramic. In one example of an unpackaged integrated circuit component 920, a single monolithic integrated circuit die comprises solder bumps attached to contacts on the die. The solder bumps allow the die to be directly attached to the interposer 904. The integrated circuit component 920 can comprise one or more computing system components, such as one or more processor units (e.g., system-on-a-chip (SoC), processor core, graphics processor unit (GPU), accelerator, chipset processor), I/O controller, memory, or network interface controller. In some embodiments, the integrated circuit component 920 can comprise one or more additional active or passive devices such as capacitors, decoupling capacitors, resistors, inductors, fuses, diodes, transformers, sensors, electrostatic discharge (ESD) devices, and memory devices.

In embodiments where the integrated circuit component 920 comprises multiple integrated circuit dies, they dies can be of the same type (a homogeneous multi-die integrated circuit component) or of two or more different types (a heterogeneous multi-die integrated circuit component). A multi-die integrated circuit component can be referred to as a multi-chip package (MCP) or multi-chip module (MCM).

In addition to comprising one or more processor units, the integrated circuit component 920 can comprise additional components, such as embedded DRAM, stacked high bandwidth memory (HBM), shared cache memories, input/output (I/O) controllers, or memory controllers. Any of these additional components can be located on the same integrated circuit die as a processor unit, or on one or more integrated circuit dies separate from the integrated circuit dies comprising the processor units. These separate integrated circuit dies can be referred to as “chiplets”. In embodiments where an integrated circuit component comprises multiple integrated circuit dies, interconnections between dies can be provided by the package substrate, one or more silicon interposers, one or more silicon bridges embedded in the package substrate (such as Intel® embedded multi-die interconnect bridges (EMIBs)), or combinations thereof.

Generally, the interposer 904 may spread connections to a wider pitch or reroute a connection to a different connection. For example, the interposer 904 may couple the integrated circuit component 920 to a set of ball grid array (BGA) conductive contacts of the coupling components 916 for coupling to the circuit board 902. In the embodiment illustrated in FIG. 9, the integrated circuit component 920 and the circuit board 902 are attached to opposing sides of the interposer 904; in other embodiments, the integrated circuit component 920 and the circuit board 902 may be attached to a same side of the interposer 904. In some embodiments, three or more components may be interconnected by way of the interposer 904.

In some embodiments, the interposer 904 may be formed as a PCB, including multiple metal layers separated from one another by layers of dielectric material and interconnected by electrically conductive vias. In some embodiments, the interposer 904 may be formed of an epoxy resin, a fiberglass-reinforced epoxy resin, an epoxy resin with inorganic fillers, a ceramic material, or a polymer material such as polyimide. In some embodiments, the interposer 904 may be formed of alternate rigid or flexible materials that may include the same materials described above for use in a semiconductor substrate, such as silicon, germanium, and other group III-V and group IV materials. The interposer 904 may include metal interconnects 908 and vias 910, including but not limited to through hole vias 910-1 (that extend from a first face 950 of the interposer 904 to a second face 954 of the interposer 904), blind vias 910-2 (that extend from the first or second faces 950 or 954 of the interposer 904 to an internal metal layer), and buried vias 910-3 (that connect internal metal layers).

In some embodiments, the interposer 904 can comprise a silicon interposer. Through silicon vias (TSV) extending through the silicon interposer can connect connections on a first face of a silicon interposer to an opposing second face of the silicon interposer. In some embodiments, an interposer 904 comprising a silicon interposer can further comprise one or more routing layers to route connections on a first face of the interposer 904 to an opposing second face of the interposer 904.

The interposer 904 may further include embedded devices 914, including both passive and active devices. Such devices may include, but are not limited to, capacitors, decoupling capacitors, resistors, inductors, fuses, diodes, transformers, sensors, electrostatic discharge (ESD) devices, and memory devices. More complex devices such as radio frequency devices, power amplifiers, power management devices, antennas, arrays, sensors, and microelectromechanical systems (MEMS) devices may also be formed on the interposer 904. The package-on-interposer structure 936 may take the form of any of the package-on-interposer structures known in the art. In embodiments where the interposer is a non-printed circuit board

The integrated circuit device assembly 900 may include an integrated circuit component 924 coupled to the first face 940 of the circuit board 902 by coupling components 922. The coupling components 922 may take the form of any of the embodiments discussed above with reference to the coupling components 916, and the integrated circuit component 924 may take the form of any of the embodiments discussed above with reference to the integrated circuit component 920.

The integrated circuit device assembly 900 illustrated in FIG. 9 includes a package-on-package structure 934 coupled to the second face 942 of the circuit board 902 by coupling components 928. The package-on-package structure 934 may include an integrated circuit component 926 and an integrated circuit component 932 coupled together by coupling components 930 such that the integrated circuit component 926 is disposed between the circuit board 902 and the integrated circuit component 932. The coupling components 928 and 930 may take the form of any of the embodiments of the coupling components 916 discussed above, and the integrated circuit components 926 and 932 may take the form of any of the embodiments of the integrated circuit component 920 discussed above. The package-on-package structure 934 may be configured in accordance with any of the package-on-package structures known in the art.

FIG. 10 is a block diagram of an example electrical device 1000 that may include one or more of the embodiments disclosed herein. For example, any suitable ones of the components of the electrical device 1000 may include one or more integrated circuits with frontside feedthrough connections (e.g., semiconductor devices 100, 200, IC die 400), integrated circuit device assemblies 900, integrated circuit components 920, integrated circuit devices 700, or integrated circuit dies 602 disclosed herein. A number of components are illustrated in FIG. 10 as included in the electrical device 1000, but any one or more of these components may be omitted or duplicated, as suitable for the application. In some embodiments, some or all of the components included in the electrical device 1000 may be attached to one or more motherboards, mainboards, or system boards. In some embodiments, one or more of these components are fabricated onto a single system-on-a-chip (SoC) die.

Additionally, in various embodiments, the electrical device 1000 may not include one or more of the components illustrated in FIG. 10, but the electrical device 1000 may include interface circuitry for coupling to the one or more components. For example, the electrical device 1000 may not include a display device 1006, but may include display device interface circuitry (e.g., a connector and driver circuitry) to which a display device 1006 may be coupled. In another set of examples, the electrical device 1000 may not include an audio input device 1024 or an audio output device 1008, but may include audio input or output device interface circuitry (e.g., connectors and supporting circuitry) to which an audio input device 1024 or audio output device 1008 may be coupled.

The electrical device 1000 may include one or more processor units 1002 (e.g., one or more processor units). As used herein, the terms “processor unit”, “processing unit” or “processor” may refer to any device or portion of a device that processes electronic data from registers and/or memory to transform that electronic data into other electronic data that may be stored in registers and/or memory. The processor unit 1002 may include one or more digital signal processors (DSPs), application-specific integrated circuits (ASICs), central processing units (CPUs), graphics processing units (GPUs), general-purpose GPUs (GPGPUs), accelerated processing units (APUs), field-programmable gate arrays (FPGAs), neural network processing units (NPUs), data processor units (DPUs), accelerators (e.g., graphics accelerator, compression accelerator, artificial intelligence accelerator), controller cryptoprocessors (specialized processors that execute cryptographic algorithms within hardware), server processors, controllers, or any other suitable type of processor units. As such, the processor unit can be referred to as an XPU (or xPU).

The electrical device 1000 may include a memory 1004, which may itself include one or more memory devices such as volatile memory (e.g., dynamic random access memory (DRAM), static random-access memory (SRAM)), non-volatile memory (e.g., read-only memory (ROM), flash memory, chalcogenide-based phase-change non-voltage memories), solid state memory, and/or a hard drive. In some embodiments, the memory 1004 may include memory that is located on the same integrated circuit die as the processor unit 1002. This memory may be used as cache memory (e.g., Level 1 (L1), Level 2 (L2), Level 3 (L3), Level 4 (L4), Last Level Cache (LLC)) and may include embedded dynamic random access memory (eDRAM) or spin transfer torque magnetic random access memory (STT-MRAM).

In some embodiments, the electrical device 1000 can comprise one or more processor units 1002 that are heterogeneous or asymmetric to another processor unit 1002 in the electrical device 1000. There can be a variety of differences between the processing units 1002 in a system in terms of a spectrum of metrics of merit including architectural, microarchitectural, thermal, power consumption characteristics, and the like. These differences can effectively manifest themselves as asymmetry and heterogeneity among the processor units 1002 in the electrical device 1000.

In some embodiments, the electrical device 1000 may include a communication component 1012 (e.g., one or more communication components). For example, the communication component 1012 can manage wireless communications for the transfer of data to and from the electrical device 1000. The term “wireless” and its derivatives may be used to describe circuits, devices, systems, methods, techniques, communications channels, etc., that may communicate data through the use of modulated electromagnetic radiation through a nonsolid medium. The term “wireless” does not imply that the associated devices do not contain any wires, although in some embodiments they might not.

The communication component 1012 may implement any of a number of wireless standards or protocols, including but not limited to Institute for Electrical and Electronic Engineers (IEEE) standards including Wi-Fi (IEEE 802.11 family), IEEE 802.16 standards (e.g., IEEE 802.16-2005 Amendment), Long-Term Evolution (LTE) project along with any amendments, updates, and/or revisions (e.g., advanced LTE project, ultra mobile broadband (UMB) project (also referred to as “3GPP2”), etc.). IEEE 802.16 compatible Broadband Wireless Access (BWA) networks are generally referred to as WiMAX networks, an acronym that stands for Worldwide Interoperability for Microwave Access, which is a certification mark for products that pass conformity and interoperability tests for the IEEE 802.16 standards. The communication component 1012 may operate in accordance with a Global System for Mobile Communication (GSM), General Packet Radio Service (GPRS), Universal Mobile Telecommunications System (UMTS), High Speed Packet Access (HSPA), Evolved HSPA (E-HSPA), or LTE network. The communication component 1012 may operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). The communication component 1012 may operate in accordance with Code Division Multiple Access (CDMA), Time Division Multiple Access (TDMA), Digital Enhanced Cordless Telecommunications (DECT), Evolution-Data Optimized (EV-DO), and derivatives thereof, as well as any other wireless protocols that are designated as 3G, 4G, 5G, and beyond. The communication component 1012 may operate in accordance with other wireless protocols in other embodiments. The electrical device 1000 may include an antenna 1022 to facilitate wireless communications and/or to receive other wireless communications (such as AM or FM radio transmissions).

In some embodiments, the communication component 1012 may manage wired communications, such as electrical, optical, or any other suitable communication protocols (e.g., IEEE 802.3 Ethernet standards). As noted above, the communication component 1012 may include multiple communication components. For instance, a first communication component 1012 may be dedicated to shorter-range wireless communications such as Wi-Fi or Bluetooth, and a second communication component 1012 may be dedicated to longer-range wireless communications such as global positioning system (GPS), EDGE, GPRS, CDMA, WiMAX, LTE, EV-DO, or others. In some embodiments, a first communication component 1012 may be dedicated to wireless communications, and a second communication component 1012 may be dedicated to wired communications.

The electrical device 1000 may include battery/power circuitry 1014. The battery/power circuitry 1014 may include one or more energy storage devices (e.g., batteries or capacitors) and/or circuitry for coupling components of the electrical device 1000 to an energy source separate from the electrical device 1000 (e.g., AC line power).

The electrical device 1000 may include a display device 1006 (or corresponding interface circuitry, as discussed above). The display device 1006 may include one or more embedded or wired or wirelessly connected external visual indicators, such as a heads-up display, a computer monitor, a projector, a touchscreen display, a liquid crystal display (LCD), a light-emitting diode display, or a flat panel display.

The electrical device 1000 may include an audio output device 1008 (or corresponding interface circuitry, as discussed above). The audio output device 1008 may include any embedded or wired or wirelessly connected external device that generates an audible indicator, such speakers, headsets, or earbuds.

The electrical device 1000 may include an audio input device 1024 (or corresponding interface circuitry, as discussed above). The audio input device 1024 may include any embedded or wired or wirelessly connected device that generates a signal representative of a sound, such as microphones, microphone arrays, or digital instruments (e.g., instruments having a musical instrument digital interface (MIDI) output). The electrical device 1000 may include a Global Navigation Satellite System (GNSS) device 1018 (or corresponding interface circuitry, as discussed above), such as a Global Positioning System (GPS) device. The GNSS device 1018 may be in communication with a satellite-based system and may determine a geolocation of the electrical device 1000 based on information received from one or more GNSS satellites, as known in the art.

The electrical device 1000 may include other output device(s) 1010 (or corresponding interface circuitry, as discussed above). Examples of the other output device(s) 1010 may include an audio codec, a video codec, a printer, a wired or wireless transmitter for providing information to other devices, or an additional storage device.

The electrical device 1000 may include other input device(s) 1020 (or corresponding interface circuitry, as discussed above). Examples of the other input device(s) 1020 may include an accelerometer, a gyroscope, a compass, an image capture device (e.g., monoscopic or stereoscopic camera), a trackball, a trackpad, a touchpad, a keyboard, a cursor control device such as a mouse, a stylus, a touchscreen, proximity sensor, microphone, a bar code reader, a Quick Response (QR) code reader, electrocardiogram (ECG) sensor, PPG (photoplethysmogram) sensor, galvanic skin response sensor, any other sensor, or a radio frequency identification (RFID) reader.

The electrical device 1000 may have any desired form factor, such as a hand-held or mobile electrical device (e.g., a cell phone, a smart phone, a mobile internet device, a music player, a tablet computer, a laptop computer, a 2-in-1 convertible computer, a portable all-in-one computer, a netbook computer, an ultrabook computer, a personal digital assistant (PDA), an ultra mobile personal computer, a portable gaming console, etc.), a desktop electrical device, a server, a rack-level computing solution (e.g., blade, tray or sled computing systems), a workstation or other networked computing component, a printer, a scanner, a display device (e.g., monitor, television), a set-top box, an entertainment control unit, a video game console, a video playback device, a vehicle control unit, a digital camera, a digital video recorder, a wearable electrical device or an embedded computing system (e.g., computing systems that are part of a vehicle, smart home appliance, consumer electronics product or equipment, manufacturing equipment). In some embodiments, the electrical device 1000 may be any other electronic device that processes data. In some embodiments, the electrical device 1000 may comprise multiple discrete physical components. Given the range of devices that the electrical device 1000 can be manifested as in various embodiments, in some embodiments, the electrical device 1000 can be referred to as a computing device or a computing system.

While the concepts of the present disclosure are susceptible to various modifications and alternative forms, specific embodiments thereof have been shown by way of example in the drawings and are described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features. Further, it should be understood that the various embodiments shown in the figures are illustrative representations and are not necessarily drawn to scale.

Moreover, the illustrations and/or descriptions of various embodiments may be simplified or approximated for case of understanding, and as a result, they may not necessarily reflect the level of precision nor variation that may be present in actual embodiments. For example, while some figures generally indicate straight lines, right angles, and smooth surfaces, actual implementations of the disclosed embodiments may have less than perfect straight lines and right angles, and some features may have surface topography or otherwise be non-smooth, given real-world limitations of fabrication processes. Similarly, illustrations and/or descriptions of how components are arranged may be simplified or approximated for case of understanding and may vary by some margin of error in actual embodiments (e.g., due to fabrication processes, etc.).

Unless otherwise specified, the use of the ordinal adjectives “first,” “second,” and “third,” etc., to describe a common object, merely indicate that different instances of like objects to which are being referred and are not intended to imply that the objects so described must be in a given sequence, either temporally, spatially, in ranking or in any other manner.

The terms “substantially,” “close,” “approximately,” “near,” and “about,” generally refer to being within +/−10% of a target value (unless otherwise specified). Similarly, terms describing spatial relationships, such as “perpendicular,” “orthogonal,” or “coplanar,” may refer to being substantially within the described spatial relationships (e.g., within +/−10 degrees of orthogonality).

Certain terminology may also be used in the foregoing description for the purpose of reference only, and thus are not intended to be limiting. For example, terms such as “upper,” “lower,” “above,” “below,” “bottom,” and “top” refer to directions in the drawings to which reference is made. Terms such as “front,” “back,” “rear,” and “side” describe the orientation and/or location of portions of the component within a consistent but arbitrary frame of reference which is made clear by reference to the text and the associated drawings describing the component under discussion. Such terminology may include the words specifically mentioned above, derivatives thereof, and words of similar import.

The terms “over”, “under”, “between”, “adjacent”, “to”, and “on” as used herein may refer to a relative position of one layer or component with respect to other layers or components. For example, one layer “over”, “under”, or “on” another layer, “adjacent” to another layer, or bonded “to” another layer may be directly in contact with the other layer or may have one or more intervening layers. One layer “between” layers may be directly in contact with the layers or may have one or more intervening layers.

The meaning of “a,” “an,” and “the” include plural references. The meaning of “in” includes “in” and “on.”

For the purposes of the present disclosure, phrases “A and/or B” and “A or B” mean (A), (B), or (A and B). For the purposes of the present disclosure, the phrase “A, B, and/or C” means (A), (B), (C), (A and B), (A and C), (B and C), or (A, B and C).

Views labeled “cross-sectional”, “profile” and “plan” correspond to orthogonal planes within a cartesian coordinate system. For example, cross-sectional and profile views may be taken in the x-z plane or y-z plane, and plan views may be taken in the x-y plane.

The term “package” generally refers to a self-contained carrier of one or more dice, where the dice are attached to or embedded in the package substrate, and may be encapsulated for protection, with integrated or wire-bonded interconnects between the dice, along with leads, pins, or bumps located on the external portions of the package substrate. The package may contain a single die, or multiple dice, providing respective functions. The package may be mounted on a printed circuit board for interconnection with other packaged integrated circuits and discrete components, forming a larger circuit.

The term “cored” generally refers to a substrate of an integrated circuit package built upon a board, card, or wafer comprising a non-flexible stiff material. Typically, a small printed circuit board is used as a core, upon which integrated circuit device and discrete passive components may be soldered. Typically, the core has vias extending from one side to the other, allowing circuitry on one side of the core to be coupled directly to circuitry on the opposite side of the core. The core may also serve as a platform for building up layers of conductors and dielectric materials.

The term “coreless” generally refers to a substrate of an integrated circuit package having no core. The lack of a core may allow for higher-density package architectures, as the through-vias may have relatively large dimensions and pitch compared to high-density interconnects.

The term “land side” generally refers to the side of the substrate of the integrated circuit package closest to the plane of attachment to a printed circuit board, motherboard, or other package. This is in contrast to the term “die side”, which generally refers to the side of the substrate of the integrated circuit package to which the die or dice are attached.

The terms “dielectric” and “dielectric material” generally refer to any type or number of non-electrically conductive materials. In some cases, dielectric material may be used to make up the structure of a package substrate. For example, dielectric material may be incorporated into an integrated circuit package as layers of laminate film or as a resin molded over integrated circuit dice mounted on the substrate.

The term “metallization” generally refers to metal layers formed on, over, and/or through the dielectric material of the package substrate. The metal layers are generally patterned to form metal structures such as traces and bond pads. The metallization of a package substrate may be confined to a single layer or in multiple layers separated by layers of dielectric.

The term “bond pad” generally refers to metallization structures that terminate integrated traces and vias in integrated circuit packages and dies. The term “solder pad” may be occasionally substituted for “bond pad” and may carry the same or similar meaning.

The term “bump” generally refers to a conductive layer or structure formed on a bond pad, which is typically made of solder or metal and has a round or curved shape, hence the term “bump”.

The term “substrate” generally refers to a planar platform that may include dielectric, metallization, and/or semiconductor device structures. For example, a substrate may be or may contain an integrated circuit die or may be used to fabricate an integrated circuit die. As another example, a substrate may mechanically support and electrically couple one or more IC dies on a single platform, with encapsulation of the one or more IC dies by a moldable dielectric material. A substrate may include bumps or pads as bonding interconnects on one or both sides. For example, one side of the substrate, generally referred to as the “die side”, may include bumps or pads for chip or die bonding. The opposite side of the substrate, generally referred to as the “land side”, may include bumps or pads for bonding the package to a printed circuit board.

The term “assembly” generally refers to a grouping of parts into a single functional unit. For example, certain parts may be permanently bonded together, integrated together, and/or mechanically assembled (e.g., where parts may be removable) into a functional unit.

The terms “coupled” or “connected” means a direct or indirect connection, such as a direct electrical, mechanical, magnetic, or fluidic connection between the things that are connected or an indirect connection, through one or more passive or active intermediary devices.

The term “circuit” or “module” may refer to one or more passive and/or active components that are arranged to cooperate with one another to provide a desired function. The term “signal” may refer to at least one current signal, voltage signal, magnetic signal, or data/clock signal.

### EXAMPLES

Illustrative examples of the technologies described throughout this disclosure are provided below. Embodiments of these technologies may include any one or more, and any combination of, the examples described below. In some embodiments, at least one of the systems or components set forth in one or more of the preceding figures may be configured to perform one or more operations, techniques, processes, and/or methods as set forth in the following examples.

Example 1 includes a semiconductor device, comprising: a first interconnect; a second interconnect; and a layer between the first and second interconnects, wherein the layer comprises a plurality of epitaxial structures and a conductive feedthrough, wherein the conductive feedthrough extends through the layer and electrically couples the first and second interconnects, wherein one or more of the epitaxial structures are truncated by the conductive feedthrough.

Example 2 includes the semiconductor device of Example 1, wherein the plurality of epitaxial structures include: a plurality of source or drain structures; and one or more partial source or drain structures, wherein the conductive feedthrough extends through the one or more partial source or drain structures, wherein the one or more partial source or drain structures are truncated by the conductive feedthrough.

Example 3 includes the semiconductor device of Example 2, wherein the one or more partial source or drain structures comprise epitaxial residue from one or more of the epitaxial structures truncated by the conductive feedthrough.

Example 4 includes the semiconductor device of any of Examples 2-3, wherein the layer further comprises a shorted transistor, wherein the shorted transistor comprises one or more of the partial source or drain structures, wherein the shorted transistor is shorted by the conductive feedthrough.

Example 5 includes the semiconductor device of any of Examples 2-4, wherein the layer further comprises one or more transistors, wherein individual transistors comprise a source contact, a drain contact, a source, a drain, a gate, and a channel, wherein the source and the drain are respective ones of the plurality of source or drain structures.

Example 6 includes the semiconductor device of Example 5, wherein the one or more transistors include one or more nanoribbon transistors, gate-all-around transistors, fin field-effect transistors, planar transistors, or two-dimensional transistors.

Example 7 includes the semiconductor device of any of Examples 1-6, wherein the plurality of epitaxial structures include: a plurality of source or drain structures; and epitaxial residue from one or more truncated source or drain structures, wherein the one or more truncated source or drain structures are truncated by the conductive feedthrough.

Example 8 includes the semiconductor device of Example 7, wherein the one or more truncated source or drain structures are adjacent to the conductive feedthrough.

Example 9 includes the semiconductor device of any of Examples 1-8, further comprising a dielectric liner on the conductive feedthrough.

Example 10 includes the semiconductor device of any of Examples 1-9, wherein the plurality of epitaxial structures include one or more p-type epitaxial structures and one or more n-type epitaxial structures, wherein: the p-type epitaxial structures comprise: silicon doped with boron; or silicon and germanium doped with boron; and the n-type epitaxial structures comprise silicon doped with phosphorous.

Example 11 includes the semiconductor device of any of Examples 1-10, wherein the conductive feedthrough comprises a metal such as tungsten, copper, cobalt, or molybdenum.

Example 12 includes the semiconductor device of Example 11, wherein the metal comprises tungsten.

Example 13 includes the semiconductor device of any of Examples 1-12, further comprising a substrate, wherein the layer is over the substrate, wherein the first interconnect is over the layer, and wherein the second interconnect is under the substrate.

Example 14 includes an electronic device, comprising: a first interconnect; a second interconnect; and a device layer between the first and second interconnects, wherein the device layer comprises a plurality of transistors and a plurality of conductive feedthroughs, wherein the conductive feedthroughs extend through some of the transistors, and wherein the conductive feedthroughs electrically couple the first and second interconnects.

Example 15 includes the electronic device of Example 14, wherein some of the transistors are truncated by the conductive feedthroughs, wherein the conductive feedthroughs extend through the truncated transistors.

Example 16 includes the electronic device of any of Examples 14-15, wherein some of the transistors are shorted by the conductive feedthroughs, wherein the conductive feedthroughs extend through the shorted transistors.

Example 17 includes the electronic device of any of Examples 14-16, wherein: the respective transistors comprise a source contact, a drain contact, a source, a drain, a gate, and a channel; and the conductive feedthroughs extend through the source contact, the drain contact, the source, the drain, the gate, and/or the channel of some of the transistors.

Example 18 includes the electronic device of any of Examples 14-16, wherein: the respective transistors comprise a source contact, a drain contact, a source, a drain, a gate, and a channel; and the conductive feedthroughs truncate the source contact, the drain contact, the source, the drain, the gate, and/or the channel of some of the transistors.

Example 19 includes the electronic device of any of Examples 14-18, further comprising dielectric liners on the conductive feedthroughs.

Example 20 includes the electronic device of any of Examples 14-19, further comprising a substrate, wherein the device layer and the first interconnect are over the substrate, and wherein the second interconnect is under the substrate.

Example 21 includes the electronic device of any of Examples 14-20, wherein the plurality of transistors include one or more nanoribbon transistors, gate-all-around transistors, fin field-effect transistors, planar transistors, or two-dimensional transistors.

Example 22 includes the electronic device of any of Examples 14-21, further comprising: a circuit board; and an integrated circuit coupled to the circuit board, wherein the integrated circuit comprises processing circuitry, memory circuitry, storage circuitry, or communication circuitry, wherein at least some of the transistors are comprised in the processing circuitry, the memory circuitry, the storage circuitry, or the communication circuitry.

Example 23 includes a method, comprising: receiving a substrate; forming a device layer over the substrate, wherein the device layer comprises a plurality of transistors; forming one or more conductive feedthroughs in the device layer, wherein one or more of the transistors are truncated by the conductive feedthroughs; forming a first interconnect over the device layer; and forming a second interconnect under the device layer, wherein the second interconnect is electrically coupled to the first interconnect via the conductive feedthroughs.

Example 24 includes the method of Example 23, wherein: the method is a method of forming an integrated circuit, wherein the integrated circuit comprises the device layer, the one or more conductive feedthroughs, the first interconnect, and the second interconnect; and forming the one or more conductive feedthroughs in the device layer comprises: etching one or more holes in the device layer; and filling the holes with a conductive material.

Example 25 includes the method of Example 24, wherein forming the one or more conductive feedthroughs in the device layer further comprises: before filling the holes with the conductive material, forming a dielectric liner on an interior surface of the respective holes.

Example 26 includes the method of any of Examples 24-25, wherein forming the one or more conductive feedthroughs in the device layer further comprises: after filling the holes with the conductive material, polishing the conductive material.

Example 27 includes the method of any of Examples 24-26, wherein the conductive material comprises a metal.

Example 28 includes the method of Example 27, wherein the metal comprises tungsten.

Example 29 includes the method of any of Examples 23-28, wherein the conductive feedthroughs extend through one or more of the transistors.

Example 30 includes the method of any of Examples 23-29, wherein: the respective transistors comprise a source contact, a drain contact, a source, a drain, a gate, and a channel; and the conductive feedthroughs truncate the source contact, the drain contact, the source, the drain, the gate, and/or the channel of one or more of the transistors.

Example 31 includes the method of any of Examples 23-30, wherein the substrate comprises silicon.

1. A semiconductor device, comprising:
a first interconnect;
a second interconnect; and
a layer between the first and second interconnects, wherein the layer comprises a plurality of epitaxial structures and a conductive feedthrough, wherein the conductive feedthrough extends through the layer and electrically couples the first and second interconnects, wherein one or more of the epitaxial structures are truncated by the conductive feedthrough.

2. The semiconductor device of claim 1, wherein the plurality of epitaxial structures include:
a plurality of source or drain structures; and
one or more partial source or drain structures, wherein the conductive feedthrough extends through the one or more partial source or drain structures, wherein the one or more partial source or drain structures are truncated by the conductive feedthrough.

3. The semiconductor device of claim 2, wherein the layer further comprises a shorted transistor, wherein the shorted transistor comprises one or more of the partial source or drain structures, wherein the shorted transistor is shorted by the conductive feedthrough.

4. The semiconductor device of claim 2, wherein the layer further comprises one or more transistors, wherein individual transistors comprise a source contact, a drain contact, a source, a drain, a gate, and a channel, wherein the source and the drain are respective ones of the plurality of source or drain structures.

5. The semiconductor device of claim 1, further comprising a dielectric liner on the conductive feedthrough.

6. The semiconductor device of claim 1, wherein the plurality of epitaxial structures include one or more p-type epitaxial structures and one or more n-type epitaxial structures, wherein:
the p-type epitaxial structures comprise:
silicon doped with boron; or
silicon and germanium doped with boron; and
the n-type epitaxial structures comprise silicon doped with phosphorous.

7. The semiconductor device of claim 1, wherein the conductive feedthrough comprises tungsten, copper, cobalt, or molybdenum.

8. An electronic device, comprising:
a first interconnect;
a second interconnect; and
a device layer between the first and second interconnects, wherein the device layer comprises a plurality of transistors and a plurality of conductive feedthroughs, wherein the conductive feedthroughs extend through some of the transistors, and wherein the conductive feedthroughs electrically couple the first and second interconnects.

9. The electronic device of claim 8, wherein some of the transistors are truncated by the conductive feedthroughs, wherein the conductive feedthroughs extend through the truncated transistors.

10. The electronic device of claim 8, wherein some of the transistors are shorted by the conductive feedthroughs, wherein the conductive feedthroughs extend through the shorted transistors.

11. The electronic device of claim 8, wherein:
the respective transistors comprise a source contact, a drain contact, a source, a drain, a gate, and a channel; and
the conductive feedthroughs extend through the source contact, the drain contact, the source, the drain, the gate, and/or the channel of some of the transistors.

12. The electronic device of claim 8, further comprising dielectric liners on the conductive feedthroughs.

13. The electronic device of claim 8, further comprising a substrate, wherein the device layer and the first interconnect are over the substrate, and wherein the second interconnect is under the substrate.

14. The electronic device of claim 8, wherein the plurality of transistors include one or more nanoribbon transistors, gate-all-around transistors, fin field-effect transistors, planar transistors, or two-dimensional transistors.

15. The electronic device of claim 8, further comprising:
a circuit board; and
an integrated circuit coupled to the circuit board, wherein the integrated circuit comprises processing circuitry, memory circuitry, storage circuitry, or communication circuitry, wherein at least some of the transistors are comprised in the processing circuitry, the memory circuitry, the storage circuitry, or the communication circuitry.

16. A method, comprising:
receiving a substrate;
forming a device layer over the substrate, wherein the device layer comprises a plurality of transistors;
forming one or more conductive feedthroughs in the device layer, wherein one or more of the transistors are truncated by the conductive feedthroughs;
forming a first interconnect over the device layer; and
forming a second interconnect under the device layer, wherein the second interconnect is electrically coupled to the first interconnect via the conductive feedthroughs.

17. The method of claim 16, wherein:
the method is a method of forming an integrated circuit, wherein the integrated circuit comprises the device layer, the one or more conductive feedthroughs, the first interconnect, and the second interconnect; and
forming the one or more conductive feedthroughs in the device layer comprises:
etching one or more holes in the device layer; and
filling the holes with a conductive material.

18. The method of claim 17, wherein forming the one or more conductive feedthroughs in the device layer further comprises:
before filling the holes with the conductive material, forming a dielectric liner on an interior surface of the respective holes.

19. The method of claim 17, wherein forming the one or more conductive feedthroughs in the device layer further comprises:
after filling the holes with the conductive material, polishing the conductive material.

20. The method of claim 16, wherein:
the respective transistors comprise a source contact, a drain contact, a source, a drain, a gate, and a channel; and
the conductive feedthroughs truncate the source contact, the drain contact, the source, the drain, the gate, and/or the channel of one or more of the transistors.
